(19) United States
(12) Patent Application Publication
(10) Pub. No.: US 2024/0092750 A1
(43) Pub. Date: Mar. 21, 2024

(54) AQUEOUS FORMULATIONS OF CYTOTOXIC TAXANES WITH CYCLODEXTRIN

(71) Applicant: Northwestern University, Evanston, IL (US)

(72) Inventors: Nathan C. GIANNESCHI, Wilmette, IL (US); Brittney Elaine WILLIAMS, Evanston, IL (US); Matthew Peter THOMPSON, Evanston, IL (US)

(21) Appl. No.: 18/237,112

(22) Filed: Aug. 23, 2023

Related U.S. Application Data
(60) Provisional application No. 63/400,148, filed on Aug. 23, 2022.

(51) Int. Cl. C07D 305/14; A61K 45/06; A61K 47/40; A61P 35/00

(52) U.S. Cl. CPC C07D 305/14; A61K 45/06; A61K 47/40; A61P 35/00

(57) ABSTRACT

Provided in the present disclosure are complexes and solutions demonstrating improved aqueous solubility of derivatized cytotoxic taxane drug moieties. In some other aspects, the disclosure provides methods of using such complexes and solutions for the treatment of cancer. In some further aspects, the disclosure provides combination therapies that may be suitable when used in combination with the use of the complexes disclosed herein.

### CROSS-REFERENCE TO RELATED APPLICATIONS

This application claims the benefit of priority to U.S. Provisional Patent Application No. 63/400,148, filed Aug. 23, 2022, which is hereby incorporated by reference in its entirety.

### BACKGROUND OF INVENTION

Breast cancer remains one of the leading causes of death among women worldwide; however, progress has been shown with the advent of combination cytotoxin/immunotherapy therapeutic strategies. In light of this, there is an urgent need for simpler and more efficient methods of delivering classical cytotoxins and/or immunotherapeutics to realize the potential of these novel combination therapies.

Human Serum Albumin (HSA) is the most prevalent protein in human blood. Using this protein as a carrier for drug delivery is a commonly used strategy with utility in significant extension of half-life. In chemotherapeutic cytotoxin delivery, HSA has famously been used in a nanoparticle formulation of paclitaxel, ABRAXANE™. ABRAXANE™ is a polydisperse amorphous paclitaxel (PTX) formulation that consists of micro- and nanoscale particulates of the drug with HSA. This formulation was approved by the U.S. Food and Drug Administration (FDA) in 2005 because it avoided the use of toxic excipients, which are found in the standard TAXOL™ formulation of paclitaxel, namely CREMOPHOR™ EL. In initial preclinical studies and over the years, ABRAXANE™ has been shown to have a modest therapeutic index, comparable to TAXOL™, and a maximum tolerated dose (MTD) in the range of 20-30 mg/kg in mice. With the advent of combination immunotherapeutics using ABRAXANE™, it is evident that paclitaxel formulations with a vastly, increased therapeutic index would be highly beneficial.

Previous efforts for improved cytotoxic taxane formulations involves covalently linking paclitaxel (PTX) with 1,18-octadecanedioic acid (ODDA) at the 2′ position of the taxane. This derivate is then complexed with HSA prior to intravenous injection. See, C. E. Callmann et al. “Antitumor Activity of 1,18-Octadecanedioic Acid-Paclitaxel Complexed with Human Serum Albumin,” J. Am. Chem. Soc. 2019, 141(3)L11765-11769, which is incorporated by reference herein in its entirety for all purposes, but particularly for descriptions of preparation, use and characterization of derivates of taxanes and taxane formulations for treatment of cancer, to the extent not inconsistent with the present disclosure. The complex takes advantage of five high affinity long chain, fatty acid (LCFA) binding sites present on native HSA, which allow for five ODDA-PTX molecules to be bound to each albumin protein. Important aspects of such formulations include vastly improved therapeutic index believed to be due to targeting to fatty acid transporters in cancer tissue and prolonged circulation half-life.

Published PCT application WO2021/007322 reports methods for using prodrugs of small molecule cytotoxins for treatment of cancer. Methods include, among others, use of prodrugs of the cytotoxic taxane paclitaxel in which the paclitaxel is modified (derivatized) at the 2′ position with a hydrophobic group linked to a carboxylic acid group, a carboxylate anion or a carboxylate ester. This published application is incorporated by reference herein in its entirety for all purposes, but particularly for descriptions of preparation, use and characterization of the derivative of paclitaxel and formulations for treatment of cancer containing paclitaxel, to the extent not inconsistent with the present disclosure.

Despite the success of ODDA-PTX in animal studies, a major issue is the need for preformulation with HSA prior to injection. This is needed because ODDA-PTX is not soluble in water alone, limiting further animal testing capabilities. Thus, further development of water soluble formulations of paclitaxel and other taxanes are of significant interest in the art.

### SUMMARY OF THE INVENTION

In an aspect, the disclosure provides an inclusion complex characterized by formula (FX1):

(A¹—X¹—X²—T²)m:(β-CyD)n (FX1)

or a salt thereof, wherein: A¹ is a carboxylic acid group, a carboxylate anion, or a carboxylate ester; T² is a cytotoxic taxane drug moiety, which has a molecular weight of no more than 1600 Da; X¹ is an optionally substituted alkylene group having 8-22 carbon atoms; X² is a direct bond, an organic group moiety, —O—C(═O), —O—C(═O)—O—, —C(═O)—, —O—, —S—, —S(═O)—, —S(═O)₂—, —S—S—, —N═, ═N—, —N(H)—, —N═N—N(H)—, —N(H)—N═N—, —N(OH)—, or —N(═O)—; β-CyD is a water-soluble beta-cyclodextrin; m is the number of derivatized cytotoxic taxanes associated with the beta-cyclodextrin in the inclusion complex; and n is the number of beta-cyclodextrin associated with the derivatized cytotoxic taxane drug moiety in the inclusion complex; wherein the ratio of m:n ranges from 1:1 to 3:10.

Also disclosed herein is an aqueous pharmaceutically acceptable solution comprising: an aqueous carrier; and one or more than one of the inclusion complexes disclosed herein, or salt thereof, wherein the total concentration of the cytotoxic taxane drug moiety in the aqueous pharmaceutically acceptable solution is equal to 0.5 mg/mL or higher.

In a further aspect, the disclosure provides a method of treating cancer, the method comprising administering to a subject a therapeutically effective amount of one or more of the inclusion complexes disclosed herein, or any of the aqueous pharmaceutically acceptable solutions described herein.

In a further aspect, the disclosure provides a method for preparation of an improved pharmaceutically acceptable aqueous solution containing 9.5 mM or greater of a taxane moiety, which comprises the steps of: preparing any of the inclusion complexes disclosed herein by mixing A¹—X¹—X²—T² with the β-CyD wherein the molar ratio of β-CyD to A¹—X¹—X²—T² is greater than 1 to form one or more inclusion complexes; and dissolving the inclusion complex in a selected pharmaceutically acceptable aqueous solution at a concentration equal to or greater than 9.5 mM with respect to the taxane drug moiety.

Also disclosed herein, is an aspect providing a method of generating any of the inclusion complexes disclosed herein, the method comprising the steps of: providing a solvent; dissolving A¹—X¹—X²—T² in the solvent to form a solution; preparing an aqueous β-CyD by dissolving β-CyD in water; combing the aqueous β-CyD and the solution wherein the molar ratio of β-CyD to A¹—X¹—X²—T² is greater than 1 to form a mixture; and removing the solvent from the mixture; wherein the removing step results in the formation of a solid, thereby generating the inclusion complex.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1: Synthetic Scheme of ODDA-PTX. The illustrated method is exemplary and can be readily employed without undue experimentation to synthesize analogous 2′ derivatives (prodrugs) of other cytotoxic taxanes with any optionally substituted α,ω-dicarboxylic acid.

FIG. 2: Deprotection of TIPS from PTX-ODDA-TIPS. The illustrated method is exemplary and can be readily employed without undue experimentation to synthesize analogous 2′ derivatives (prodrugs) of other cytotoxic taxanes with any optionally substituted α,ω-dicarboxylic acid.

FIG. 3: Structure of Heptakis β-cyclodextrin (“Hep β-CyD”). Structures of a number of other water-soluble β-cyclodextrins are known in the art.

FIG. 4A-4C: In vitro tubulin polymerization assay to confirm prodrug status of ODDA-PTX is maintained in the β-cyclodextrin inclusion formulation. FIG. 4A depicts the optical density (nm) of 1 mM GTP. FIG. 4B depicts the optical density (nm) of 10 μM Paclitaxel. FIG. 4C depicts the optical density (nm) of 10 μM Hep β-CyD:ODDA-PTX.

FIG. 5: Retention times of ODDA-PTX:Hep β-CyD, ODDA-PTX, and cyclodextrin in a 0 to 100% Buffer B gradient over 30 minutes.

FIG. 6: Effect of ODDA-PTX-Hep β-CyD on HSA folding as a function of molar ratio.

FIG. 7: Isothermal calorimetry data showing the binding of ODDA-PTX:Hep β-Cyclodextrin to Human Serum Albumin.

FIG. 8: Cell viability in HT-1080, tissue cancer, as a function of addition of ODDA-PTX:Hep β-CyD.

FIG. 9: Cell viability in MCF-7, triple negative breast cancer, as a function of addition of PTX, ODDA-PTX, or ODDA-PTX:β-CyD. The asterisks (*) in the legend refer to samples which were first solubilized in DMSO then diluted in buffer due to the insolubility of PTX and ODDA-PTX in the buffer alone.

FIG. 10: Schematic graphic depicting a bifunctionalized prodrug binding with albumin.

FIGS. 11A-11B: FIG. 11A depicts an example of an ODDA-cytotoxic taxane structure where the cytotoxic taxane is paclitaxel. FIG. 11B depicts an example of a VTX, where the exemplary ODDA-PTX of FIG. 11A is shown as bound to HSA site 4.

FIG. 12: Example of ODDA-PTX synthesis and complexation with HSA to yield VTX.

FIG. 13: VTX sliced cross section to provide a more detailed view of the binding pocket illustrated with exemplary diacid prodrug of cytotoxic taxane. “C18” refers to a long-chain fatty acid having 18 carbons.

FIGS. 14A-14B: Results from in vivo efficacy studies in HT1080 fibrosarcoma model. FIG. 14A depicts the relative tumor volume over time for VTX 250, Abx 15, PTX15, and the saline control. FIG. 14B depicts the % survival over time for each treatment group.

FIG. 15: Graphic depicting an example of an ODDA-PTX-βCyD inclusion complex with underivatized β-CyD.

FIG. 16: Schematic graphic depicting an example of a fatty acid-CyD inclusion complex with underivatized α-CyD.

FIG. 17: Continuous variation plot depicting the complex formation of ODDA-PTX and βCyD.

FIG. 18: Transmission electron microscopy (TEM) image at 12,000× magnification depicting micelle formation of 1000 nM of ODDA-PTX-Hep β-CyD. Scale bar: 1 μm.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The following abbreviations are used herein: ESI-MS refers to electrospray ionization mass spectrometry; TEM refers to transmission electron microscopy; STEM refers to scanning TEM; CD refers to circular dichroism; MED refers to minimum effective dose; MTD refers to maximum tolerated dose; TIPS refers to triisopropyl silane; PTX refers to paclitaxel; ODDA refers to 1,18-octadecanedioic acid; LCFA refers to long-chain fatty acid; CyD refers to cyclodextrin; Hep β-CyD refers to Heptakis (2,6-di-O methyl) Beta-cyclodextrin; and HSA refers to human serum albumin.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “hydrocarbon” refers to an organic group composed of carbon and hydrogen, which can be saturated or unsaturated, and can include aromatic groups. The term “hydrocarbyl” refers to a monovalent or polyvalent (e.g., divalent or higher) hydrocarbon moiety. In some cases, a divalent hydrocarbyl group is referred to as a “hydrocarbylene” group.

As used herein, “alkyl” refers to a straight or branched chain saturated hydrocarbon having 1 to 30 carbon atoms, which may be optionally substituted, as herein further described, with multiple degrees of substitution being allowed. Examples of “alkyl,” as used herein, include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl. In some instances, the “alkyl” group can be divalent, in which case, the group can alternatively be referred to as an “alkylene” group. Also, in some instances, one or more of the carbon atoms in the alkyl or alkylene group can be replaced by a heteroatom (e.g., selected from nitrogen, oxygen, or sulfur, including N-oxides, sulfur oxides, sulfur dioxides, and carbonyl groups, where feasible), and is referred to as a “heteroalkyl” or “heteroalkylene” group, respectively. Non-limiting examples include “oxyalkyl” or “oxyalkylene” groups, which refer to groups where a carbon atom in the alkyl or alkylene group is replaced by oxygen. Non-limiting examples of oxyalkyl or oxyalkylene groups include alkyl or alkylene chains that contain a carbonyl group, and also alkoxylates, polyalkylene oxides, and the like.

The number of carbon atoms in any group or compound can be represented by the terms: “C_{z}” which refers to a group or compound having z carbon atoms, and “C_{x-y}”, which refers to a group or compound containing from x to y, inclusive, carbon atoms. For example, “C₁₋₆ alkyl” represents an alkyl group having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl (as well as all isomers thereof). The same logic applies to other types of functional groups, defined below.

As used herein, “alkenyl” refers to a straight or branched chain non-aromatic hydrocarbon having 2 to 30 carbon atoms and having one or more carbon-carbon double bonds, which may be optionally substituted, as herein further described, with multiple degrees of substitution being allowed. Examples of “alkenyl,” as used herein, include, but are not limited to, ethenyl, 2-propenyl, 2-butenyl, and 3-butenyl. In some instances, the “alkenyl” group can be divalent, in which case the group can alternatively be referred to as an “alkenylene” group. Also, in some instances, one or more of the carbon atoms in the alkenyl or alkenylene group can be replaced by a heteroatom (e.g., selected from nitrogen, oxygen, or sulfur, including N-oxides, sulfur oxides, sulfur dioxides, and carbonyl groups, where feasible), and is referred to as a “heteroalkenyl” or “heteroalkenylene” group, respectively.

As used herein, “halogen,” “halogen atom,” or “halo” refer to a fluorine, chlorine, bromine, or iodine atom. In some embodiments, the terms refer to a fluorine or a chlorine atom.

As used herein, the terms “organic group,” “organic moiety,” or “organic residue” refer to a monovalent or polyvalent functional group having at least one carbon atom, which optionally contains one or more additional atoms selected from the group consisting of hydrogen atoms, halogen atoms, nitrogen atoms, oxygen atoms, phosphorus atoms, and sulfur atoms, and which does not include covalently bound metal or semi-metal atoms. In some embodiments, these terms can include metal salts of organic groups, such as alkali metal or alkaline earth metal salts of organic anions.

As used herein, the term “pharmacophore” refers to a type of organic functional group. Standard pharmacophores are hydrophobic pharmacophores, hydrogen-bond donating pharmacophores, hydrogen-bond accepting pharmacophores, positive ionizable pharmacophores, and negative ionizable pharmacophores. The classification of organic functional groups within a compound is carried out according to standard classification systems known in the art.

As used herein, the terms “hydrophobic group,” “hydrophobic moiety,” or “hydrophobic residue” refer to an organic group which may be monovalent, divalent or multivalent that consists essentially of hydrophobic pharmacophores. In some embodiments, the terms refer to an organic group that consists of hydrophobic pharmacophores. In embodiments, a hydrophobic group includes monovalent alkyl groups and divalent alkyl group (also called alkylene groups). Such alkyl and alkylene groups are optionally substituted such that the hydrophobic nature of the group is not changed.

As used herein, the terms “hydrophilic group,” “hydrophilic moiety,” or “hydrophilic residue” refer to an organic group that comprises one pharmacophore selected from the group consisting of hydrogen bond donors, hydrogen bond acceptors, negative ionizable groups, or positive ionizable groups. In some embodiments, the terms refer to an organic group that consist essentially of pharmacophores selected from the group consisting of hydrogen bond donors, hydrogen bond acceptors, negative ionizable groups, or positive ionizable groups.

As used herein, the term “drug moiety” refers to a drug compound, or a pharmaceutically acceptable salt thereof, where an atom or a group of atoms is absent, thereby creating a monovalent or polyvalent moiety. In some embodiments, for example, a hydrogen atom is absent, thereby creating a monovalent moiety. In some other embodiments, a functional group, such as an —OH moiety, an —NH₂ moiety, or a —COOH, moiety is absent. In some embodiments, the drug moiety is a “cytotoxic drug moiety,” which refers to a drug moiety (as defined above) of a cytotoxic drug compound. In some further embodiments, the drug moiety is an “intracellularly active cytotoxic drug compound,” which refers to a cytotoxic drug moiety (defined above) whose primary cytotoxic effect occurs inside of the cell. For example, anti-folate compounds, such as gemcitabine, whose primary cytotoxic effect occurs outside of the cell (by blocking folate channels) is not intracellularly active cytotoxic drugs. One non-limiting example of a “drug moiety,” (in this case, a “paclitaxel moiety”) is the moiety of the following formula:

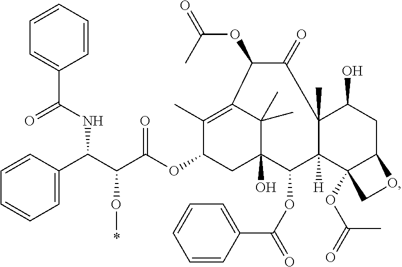 [SMILES: *O[C@@H](C(=O)O[C@H]1C[C@@]2(O)[C@@H](OC(=O)c3ccccc3)[C@H]3[C@](C)(CC(=O)[C@H](OC(C)=O)C(=C1C)C2(C)C)[C@@H](O)C[C@H]1OC[C@]13OC(C)=O)[C@@H](NC(=O)c1ccccc1)c1ccccc1]

where a hydrogen atom is absent to create a monovalent moiety that, within a compound, bonds to the rest of the molecule through the remaining oxygen atom. Note that the term “drug moiety” is not limited to any particular procedure for making such compounds.

Various methods of drawing chemical structures are used herein. In some instances, the bond line-structure method is used to depict chemical compounds or moieties. In the line-structure method, the lines represent chemical bonds, and the carbon atoms are not explicitly shown (but are implied by the intersection of the lines). The hydrogen atoms are also not explicitly shown, except in instances where they are attached to heteroatoms. Heteroatoms, however, are explicitly shown. Thus, using that methodology, the structures shown below are for 2-methylpropane, 1-methoxypropane, and 1-propanol:

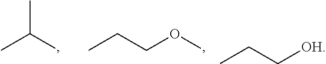

In that methodology, aromatic rings are typically represented merely by one of the contributing resonance structures. Thus, the following structures are for benzene, pyridine, and pyrrole:

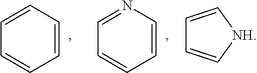

As used herein, a “protein binding moiety” is a moiety that binds non-covalently to one or more sites on a protein with a binding constant (K_{b}) of at least 100 M⁻¹ in water at 25° C.

As used herein, “administer” or “administering” means to introduce, such as to introduce to a subject a compound or composition. The term is not limited to any specific mode of delivery, and can include, for example, subcutaneous delivery, intravenous delivery, intramuscular delivery, intracisternal delivery, delivery by infusion techniques, transdermal delivery, oral delivery, nasal delivery, and rectal delivery. Furthermore, depending on the mode of delivery, the administering can be carried out by various individuals, including, for example, a health-care professional (e.g., physician, nurse, etc.), a pharmacist, or the subject (i.e., self-administration). In embodiments, administration is of a therapeutically effective amount of a pharmaceutically active ingredient (for example, an inclusion complex or a prodrug of a cytotoxic taxane, as described herein). In embodiments, administration is by intravenous delivery. In embodiments, administration is by infusion techniques.

As used herein, “treat” or “treating” or “treatment” can refer to one or more of: delaying the progress of a disease, disorder, or condition; controlling a disease, disorder, or condition; ameliorating one or more symptoms characteristic of a disease, disorder, or condition; or delaying the recurrence of a disease, disorder, or condition, or characteristic symptoms thereof, depending on the nature of the disease, disorder, or condition and its characteristic symptoms. In the context of cancer, the terms “treat” or “treating” or “treatment” can, among other things, refer to inducing apoptosis of cancerous cells, reducing the size of a cancerous tumor, delaying the growth of tumors, or inducing or enhancing an immune response against one or more cancerous cells, where the immune response has the effect of inducing apoptosis, reducing the size of a tumor, or the like.

As used herein, the term “in combination with,” such as when one compound is administered in combination with another compound, means that the two compounds are administered in a manner such that one or more biological effects of administering the first compound remain present when the second compound is administered. In embodiments, the two compounds are different types of active ingredients. In embodiments, the two active ingredients have different modes or mechanisms of action. In embodiments, administration of the two compounds in combination results in a greater than additive effect compared to the uncombined administration of the two compounds. The two compounds can, but need not, be administered in a common dosage form or at substantially the same time. For example, in the context of cancer treatment, the two compounds could be administered the same day or one or several weeks apart from each other. For example, certain small-molecule cytotoxins (or prodrugs thereof) induce an immuno-priming, whereby the small-molecule cytotoxins (or prodrugs thereof) induce cell death in a manner that tends to improve the effectiveness of subsequent treatment using an immunomodulating agent. In such instances, the two compounds may be administered “in combination with each other,” even though initial administration of the small-molecule cytotoxin to the subject may precede administration of the immunomodulating agent to the subject by several weeks or several months.

As used herein, the terms “subject” and “patient” refer to any mammal such as, but not limited to, humans, horses, cows, sheep, pigs, mice, rats, dogs, cats, and primates such as chimpanzees, gorillas, and rhesus monkeys. In some embodiments, the “subject” is a human. In some such embodiments, the “subject” is a human who exhibits one or more symptoms characteristic of a disease, disorder, or condition. The term “subject” does not require one to have any particular status with respect to a hospital, clinic, or research facility (e.g., as an admitted patient, a study participant, or the like). It will be understood that pharmaceutical formulations can be adapted for administration to different types of subjects. For example, formulations herein which include human serum albumin can be adapted, if desired, by use of serum albumin appropriate for the subject of interest.

As used herein, the term “compound” includes free acids, free bases, and salts thereof.

As used herein, the term “pharmaceutical composition” or “pharmaceutically-acceptable composition” is used to denote a composition that may be administered to a subject, e.g., orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques. In embodiments, pharmaceutical compositions are formulated for intravenous injection. In embodiments, pharmaceutical compositions are formulated for infusion.

Also included within the scope of the disclosure are the individual enantiomers of the compounds represented by formulas (FX1-FX5b), or pharmaceutically acceptable salts thereof, as well as any wholly or partially racemic mixtures thereof. The disclosure also covers the individual enantiomers of the compounds represented by formulas (FX1-FX5b), or pharmaceutically acceptable salts thereof, as well as mixtures with diastereoisomers thereof in which one or more stereocenters are inverted. Unless otherwise stated, structures depicted herein are also meant to include compounds which differ only in the presence of one or more isotopically enriched atoms. For example, compounds having the present structure, except for the replacement of a hydrogen atom by a deuterium or tritium, or the replacement of a carbon atom by a ¹³C- or ¹⁴C-enriched carbon are within the scope of the disclosure.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, “optionally” means that the subsequently described event(s) may or may not occur. In some embodiments, the optional event does not occur. In some other embodiments, the optional event does occur one or more times.

As used herein, “substituted” refers to substitution of one or more hydrogen atoms of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met. In embodiments, the number of substituents in a substituted compound or group is one. In embodiments, the number of substituents in a substituted compound or group is two. In embodiments, the number of substituents in a substituted compound or group is three. In embodiments, the number of substituents in a substituted compound or group is four.

Exemplary optional substituents include, among others, a halogen, particularly Cl or F, an oxo, a hydroxyl group, an unsubstituted alkyl group, a halogen-substituted alkyl group, an unsubstituted alkoxy group, an unsubstituted —S-alkyl group, an amino group (—NH₂), a mono- or dialkyl amino group, an alkoxy-substituted alkyl group, or a —S-alkyl substituted alkyl group. Alkyl and alkoxy groups of such substituents may include 1-6 carbon atoms or 1-3 carbon atoms. In embodiments, substituents of alkylene groups include, one or more halogens, one or more alkyl groups, one or more hydroxyl groups, one or more alkoxy groups, one or more —S-alkyl groups. In embodiments, alkyl groups on substituents are methyl groups. In embodiments, alkyl groups on substituents are ethyl groups.

As used herein, the terms “derivate,” “derivatized,” and “derivative” refer to a compound which is modified by adding one or more substituents as described herein. In embodiments, derivatization of a cytotoxic taxane forms a prodrug of the cytotoxic taxane which is activated in vivo. In embodiments, derivatization of a cyclodextrin, particularly a β-cyclodextrin, for example by partial alkylation or full alkylation of —OH groups, impacts the solubility of the β-cyclodextrin in aqueous solution. In embodiments, derivatization of a cyclodextrin, particularly a β-cyclodextrin, for example by partial alkylation or full alkylation of —OH groups, does not affect the ability of the cyclodextrin to form inclusion complexes.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present. As used herein, the phrases “consist essentially of,” “consists essentially of,” and “consisting essentially of” refer to groups that are open, but which only includes additional unnamed members that would not materially affect the basic characteristics of the claimed subject matter.

As used herein, “taxane” refers to a chemotherapeutic molecule derived from the bark of the yew tree. In embodiments, a cytotoxic taxane contains a substituted fused ring taxane core:

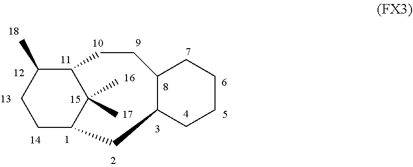

which has an ester group of structure FX4 at position 13:

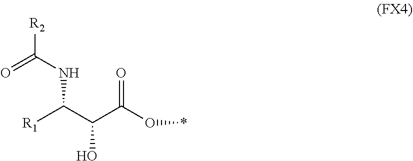

with a double bond between carbons 11-12; and various known group substitutions and stereochemical configurations at the ring positions, where a hydroxyl group is present at the 2′ carbon position of the ester group for esterification with a diacid. The prodrug lipidation and further inclusion complex with Heptakis (2,6-di-O methyl) Beta-cyclodextrin for solubilization requires the lipidation at the position 2′ carbon hydroxyl group. Specific structures of cytotoxic taxanes including, among others, paclitaxel, docetaxel, cabazitaxel, larotaxel, ortataxel, tesetaxel, and milataxel are known in the art. Additional structures of cytotoxic taxanes are described in Ren et al. (2018) Eur. J. Med. Chem. 156:692-710, which is incorporated by reference herein it in its entirety for descriptions of certain cytotoxic taxanes useful in the present invention.

“Cyclodextrins” as used herein, are macrocycles closely related to natural products from the bacterial digestion of cellulose. Cyclodextrins are cyclic oligosaccharides with 6 (α), 7 (β), or 8 (γ) glucopyranose units with a lipophilic cavity and hydrophilic outer layer. The central pore is lined with the skeletal carbons and oxygen from the glucose side chains lending to its lipophilicity. It is believed that guest molecules are essentially “trapped” inside the cavity to form an inclusion complex.

Inclusion complexes are molecular compounds having the structure of an adduct, in which a host molecule having a cavity encloses (or at least partially encloses) a guest molecule in the cavity of the host. In embodiments, the host structure is not significantly changed by the formation of the inclusion complex. Dependent upon the host molecule and the guest molecule, an inclusion complex can have different stoichiometry of guest:host. For example a given inclusion complex may have a single guest and single host (1:1 stoichiometry) or multiple guests in a single host (e.g., 2:1 guest to host). Alternatively, a single guest may associate with multiple hosts, e.g. 1:2 guest to host. Multiple guests may also be associated with one or multiple hosts, e.g., 2:1 guest to host, where two guests are at least partially enclosed with in a single host or 2:2, where two guests are enclosed within a combination of two hosts. See A. Cid-Samamed et al. (2022) “Cyclodextrins inclusion complex: Preparation methods, analytical techniques and food industry applications,” Food Chemistry 384:132467. This reference is incorporated by reference herein in its entirety for descriptions of cyclodextrins and cyclodextrin inclusion complexes.

An inclusion complex as used herein can contain one or more stoichiometric variants which are characterized by an average stoichiometric guest to host ratio. Exemplary average guest to host ratios can include 1:1.5, 2:5, 3:4, 3:7, 3:10, 4:5, 4:6, 4:7, and 4:9, among others. The inclusion complexes herein are those where the host is a cyclodextrin or derivatized cyclodextrin and the guests are certain derivatized cytotoxic taxanes. In embodiments, the host is a water-soluble beta-cyclodextrin. In embodiments, the water-soluble beta-cyclodextrin is derivatized. In embodiments, the cytotoxic taxane is derivatized at the 2′ position of the taxane with a hydrophobic linker carrying a carboxylic acid, carboxylate anion or carboxylate ester group. In embodiments, the hydrophobic linker (X¹ in Formula FX1 and FX1a) is an optionally substituted alkylene group having 8-22 carbon atoms. In embodiments, the X¹ group is an unsubstituted alkylene group. In embodiments, the X¹ group is an unsubstituted alkylene group having 14-18 carbon atoms.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, the various functional groups represented will be understood to have a point of attachment at the functional group having the hyphen or dash (-) or a dash used in combination with an asterisk (*). In other words, in the case of —CH₂CH₂CH₃ or *—CH₂CH₂CH₃, it will be understood that the point of attachment is the CH₂ group at the far left. If a group is recited without an asterisk or a dash, then the attachment point is indicated by the plain and ordinary meaning of the recited group.

As used herein, multi-atom bivalent species are to be read from left to right. For example, if the specification or claims recite A-D-E and D is defined as —OC(═O)—, the resulting group with D replaced is: A-OC(═O)-E and not A-C(═O)O-E.

Other terms are defined in other portions of this description, even though not included in this subsection.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

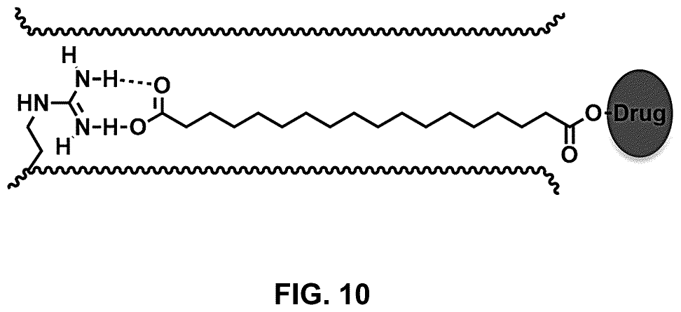

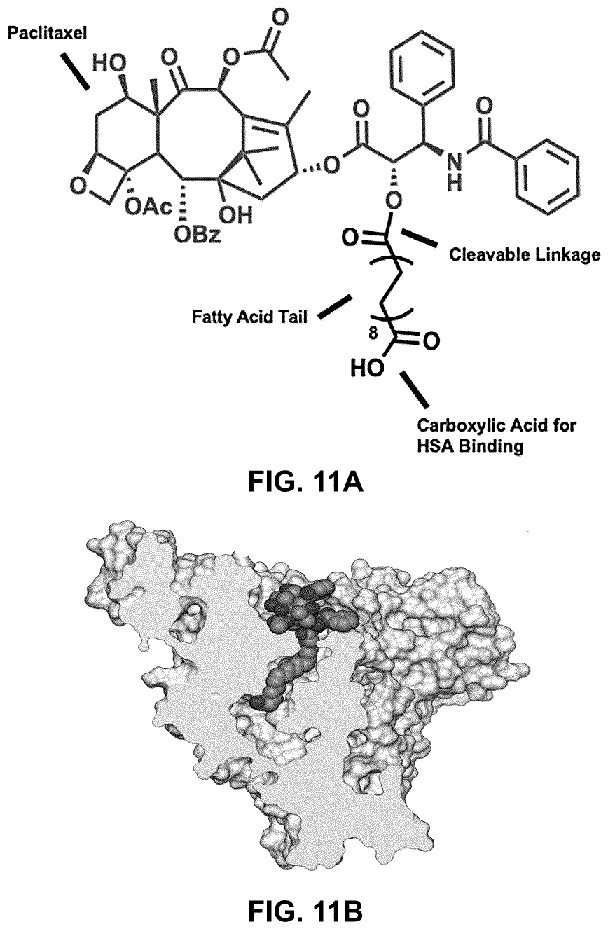

In an aspect, the invention provides an inclusion complex which is believed to harness the long-chain fatty acid (LCFA) transporter function of HSA to transport a chemotherapeutic, taxane (see, e.g., FIGS. 10 and 11B). In an aspect, the formulation of the invention uses FDA approved cyclodextrins to provide solubility to a taxane-fatty acid prodrug without the use of albumin to allow for comprehensive animal testing and clinical trials. In some embodiments, the inclusion complex comprises 1,18-octadecanedioic acid (ODDA) conjugated to a taxane. In an aspect, the taxane is paclitaxel (PTX). In keeping with this aspect, the ODDA-PTX conjugate comprises a monofunctionalized PTX ester at the 2′-hydroxyl (ODDA-PTX), as exemplified in FIG. 11A. ODDA-PTX is then mixed with HSA to yield a water-soluble complex at 5:1 prodrug:protein mol ratio (VTX) as portrayed in FIG. 11B.

In an aspect, the invention provides a β-cyclodextrin-ODDA-PTX formulated complex that improves aqueous solubility of the drug moiety via the β-cyclodextrin while minimizing off target effects through the ODDA-PTX drug. The water-soluble version of paclitaxel may provide extended half-life upon IV administration and may be targeted to tumors by hitchhiking on upregulated uptake of fatty acids, which, in-turn, may lead to lower toxicity and broad therapeutic utility. The lower toxicity may be especially useful for treatments involving adolescent subjects.

This aqueous formulation of 1,18-octadecandioic acid-paclitaxel with heptakis(2,6-di-O-methyl)-β-cyclodextrin, along with the other inclusion complexes discussed herein, have the ability to reduce the use of paclitaxel/CREMOPHOR™ EL in the clinic to treat certain cancers as a water soluble formulation. Additional features and benefits of the inventive aqueous formulations of ODDA-taxane-beta cyclodextrins will be readily apparent from the following disclosure.

Various potentially useful descriptions, background information, applications/uses of embodiments herein, terminology (to the extent not inconsistent with the terms as defined herein), mechanisms, compositions, methods, definitions, and/or other embodiments may optionally be found in, for example, U.S. Pat. No. 10,286,079, which is incorporated herein by reference to the extent not inconsistent herewith.

In some embodiments of any of the related aspects and embodiments described herein, A¹ is selected from the group consisting of a carboxylic acid group (—COOH), a carboxylate anion (—COO⁻), or a carboxylate ester (e.g., —COOR^{a}, where R^{a} is an organic group such as an alkyl or alkoxylate group). In some such embodiments, A¹ is a carboxylic acid group. In some such embodiments, A¹ is a carboxylate ester group.

In some embodiments of any of the foregoing related aspects and embodiments, X¹ is C₈₋₃₀ hydrocarbylene, which is optionally substituted. In some further embodiments, X¹ is C₈₋₂₂ hydrocarbylene, which is optionally substituted. In some further embodiments, X¹ is C₈₋₂₂ alkylene, which is optionally substituted. In some further such embodiments, X¹ is C₈₋₂₂ alkylene. In some further embodiments, X¹ is —(CH₂)₈—, —(CH₂)₁₂—, —(CH₂)₁₆—, —(CH₂)₁₈—, —(CH₂)₂₀—, or —(CH₂)₂₂—. In some other embodiments, X¹ is —(CH₂)₁₆—. In some further embodiments, X¹ is C₈₋₂₂ alkenylene, which is optionally substituted. In some further such embodiments, X¹ is C₈₋₂₂ alkenylene. In some further such embodiments, X¹ is —(CH₂)₇—CH═CH—(CH₂)₇—.

In some further embodiments of any of the foregoing related aspects or embodiments, X¹ is C₈₋₂₂ hydrocarbylene, which is optionally substituted. In some such embodiments, X¹ is C₈₋₂₂ hydrocarbylene. In some further such embodiments, X¹ is C₁₄₋₂₂ hydrocarbylene. In some further such embodiments, X¹ is C₁₆₋₂₂ hydrocarbylene. In some embodiments of any of the aforementioned embodiments, X¹ is C₁₂₋₂₂ hydrocarbylene, wherein A¹ and X² (or, if X² is a direct bond, T²) are separated from each other by at least 6, or by at least 8, or by at least 10, or by at least 12, or by at least 14, or by at least 16 carbon atoms. In some further such embodiments, X¹ is C₁₄₋₂₂ hydrocarbylene, wherein A¹ and X² (or, if X² is a direct bond, T²) are separated from each other by at least 6, or by at least 8, or by at least 10, or by at least 12, or by at least 14, carbon atoms. In some further such embodiments, X¹ is C₁₆₋₂₂ hydrocarbylene, wherein A¹ and X² (or, if X² is a direct bond, T²) are separated from each other by at least 6, or by at least 8, or by at least 10, or by at least 12, or by at least 14, or by at least 16 carbon atoms. In some further embodiments of any of the aforementioned embodiments, X¹ is C₈₋₂₂ straight-chain alkylene, X¹ is C₁₀₋₂₂ straight-chain alkylene, X¹ is C₁₂₋₂₂ straight-chain alkylene, or C₁₄₋₂₂ straight-chain alkylene, or C₁₆₋₂₂ straight-chain alkylene. In some further embodiments of any of the aforementioned embodiments, X¹ is C₈₋₂₂ straight-chain alkenylene, or C₁₀₋₂₂ straight-chain alkenylene, C₁₂₋₂₂ straight-chain alkenylene, or C₁₄₋₂₂ straight-chain alkenylene, or C₁₆₋₂₂ straight-chain alkenylene.

In some embodiments of any of the foregoing related aspects and embodiments, X² is a direct bond. In some other embodiments of any of the foregoing related aspects and embodiments, X² is an organic group moiety. In some embodiments, X² is a hydrophilic group. In some embodiments, X² is a heteroalkylene group.

In any of the aforementioned embodiments where X² is an organic group moiety, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In any of the aforementioned embodiments where X² is a heteroalkylene group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In some of the aforementioned embodiments, X² can contain certain polymer groups. Some non-limiting examples of such groups that X² can contain are polyalkylene oxide groups, such as polyethylene glycol (PEG) and various polypeptide chains.

In some embodiments, X² is an organic group selected from the group consisting of: —C(═O)—, —C≡C—, —C(H)═C(H)—, —C(═O)—O—, —O—C(═O)—, —C(═O)—NH—, —NH—C(═O)—, —NH—C(═O)—O—, —O—(C═O)—NH—, —O—C(═O)—O—, —C(═N—NH₂)—, —C(═N—R^{b})—(where R^{b} is a hydrogen atom or an alkyl group), —C(═N—OH)—, —NH—C(═O)—NH—, —NH—C(═S)—NH—, —NH—C(═S)—O—, —O—C(═S)—NH—, —NH—C(═O)—S—, —S—C(═O)—NH—, —NH—C(═S)—S—, —S—C(═S)—NH—, and the cyclic structures shown below:

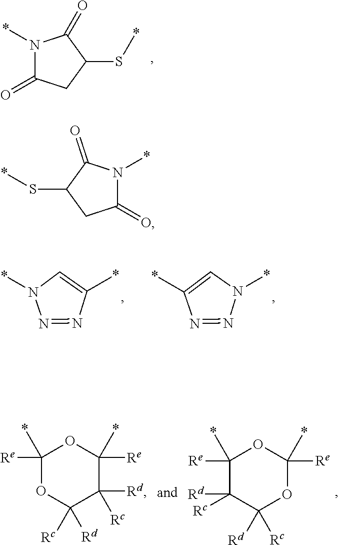

where R^{c}, R^{d}, and R^{e} are, independently at each occurrence, a hydrogen atom or C₁₋₁₀ alkyl. In some further embodiments, X² is selected from the group consisting of —O—C(═O), —O—C(═O)—O—, —C(═O)—, or —O—. In some further embodiments, X² is —C(═O)—.

In some embodiments, X² is a group selected from the group consisting of —O—, —S—, —S(═O)—, —S(═O)₂—, —S—S—, —N═, ═N—, —N(H)—, —N═N—N(H)—, —N(H)—N═N—, —N(OH)—, and —N(O)—.

In some embodiments, X² comprises one or more moieties selected from the group consisting of: —C(═O)—, —O—C(═O)—, —NH—C(═O)—, one or more moieties formed from alkylene glycols, one or more units formed from alkanol amines, one or more units formed from amino acids, and one or more units formed from hydroxyl acids. Thus, in some embodiments, X² comprises one or more moieties formed from alkylene glycols, such as a short poly(ethylene glycol) chain having 1 to 25 ethylene glycol units. In some embodiments, X² comprises one or more moieties formed from amino acids, such as an oligopeptide chain having 1 to 25 amino acid units. In some embodiments, X² comprises one or more moieties formed from hydroxy acids, such as moieties formed from glycolic acid, lactic acid, or caprolactone. In some embodiments, X² comprises a combination of a poly(ethylene glycol) chain having 1 to 25 ethylene glycol units and an oligopeptide having 1 to 25 amino acid units, and optionally one or more units formed from hydroxy acids.

In any of the above embodiments, the selection of X² will depend on the type of functional group through which it is linked to the cytotoxic taxane drug moiety, so as to avoid making compounds that are chemically unstable or impossible. The skilled artisan will be able to select combinations of X² and T² that result in chemically stable compounds, which are compounds in which the chemical structure is not substantially altered when kept at a temperature from about −80° C. to about +40° C., in the absence of moisture or other chemically reactive conditions, for at least a week.

In some embodiments of any of the foregoing related aspects and embodiments, T² can be any suitable cytotoxic taxane drug moiety. In some embodiments, the cytotoxic drug moiety is a small-molecule drug moiety, such as a cytotoxic taxane drug moiety having a molecular weight of or no more than 1600 Da, or no more than 1500 Da, or no more than 1400 Da, or no more than 1300 Da, no more than 1200 Da, or no more than 1100 Da, or no more than 1000 Da, or no more than 900 Da. In some embodiments, the cytotoxic drug moiety is an intracellularly active cytotoxic taxane drug moiety.

In some embodiments of any of the forgoing and related aspects and embodiments, the cytotoxic taxane useful in the methods and compositions herein has formula FX5:

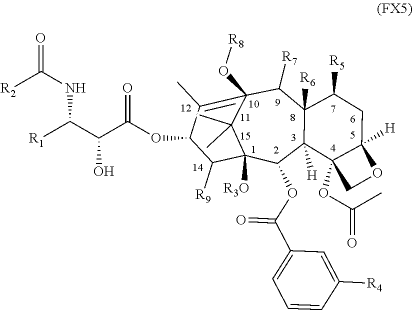

and salts thereof, wherein:
R₁ is an optionally substituted phenyl group; an optionally substituted alkyl group having 1-6 carbon atoms, particularly an unsubstituted butyl group; an optionally substituted alkenyl group having 1-6 carbon atoms, particularly an unsubstituted butenyl group; an optionally substituted furanyl group, particularly an unsubstituted furanyl group; or an optionally substituted pyridinyl group, particularly a 3-substituted pyridinyl group; and more specifically is an unsubstituted phenyl group, a 2-methylpropyl group, a 2-methylpropl-en-1-yl group; a furan-2-yl group; or a 3-fluoro-pryidin-2-yl group;
R₂ is an optionally substituted phenyl ring; an alkoxy group having 3-6 carbon atoms, particularly a butoxy group; or an alkenyl group having 3-6 carbon atoms, particularly a propenyl or butenyl group; and more specifically is an unsubstituted phenyl; a 1,1-dimethylethyoxy group (also called a t-butoxy group); a

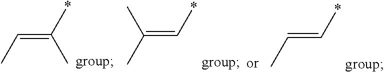

R₃ and R₉ are both H; or
R₃ and R₉ together with the atoms to which they are bonded form an ethylene carbonate ring across positions 1 and 14 of the taxane ring system, where the ethylene carbonate ring is:

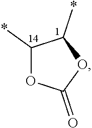

more specifically R₃ and R₉ are both H;
R₄ is H, halogen; an N₃ group; an alkyl group having 1-3 carbon atoms; an alkoxy group having 1-3 carbon atoms; a CN group; a CF₃ group, or a OCF₃ group, more specifically R₄ is H;
R₅ is H, OH or a —O—C(═O)-alkyl group where the alkyl group has 1-3 carbon atoms, or more specifically is —O—C(═O)CH₃ or —OC(═O)—C₂H₅, and
R₆ is a CH₃ group, or
R₅ and R₆ together with the atoms to which they are attached form a fused 3-membered carbon ring:

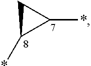

more specifically R₅ is OH and R₆ is a CH₃ group;
R₇ is ═O (to form a carbonyl group at ring position 9); and
R₈ is H, an alkyl group having 1-3 carbon atoms; an acyl group having 1-3 carbon atoms, and more specifically R₈ is H, CH₃ or an acetyl group; or
R₇ and R₈ together with the atoms to which they are attached form a substituted dioxolane ring, particularly a 1,3-dioxolane ring and more specifically a 2-substituted 1,3-dioxolane ring where the 2-substituent is an N,N-dimethylaminomethyl group:

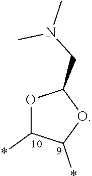

In specific embodiments, R₁-R₉ groups of formula FX5 are those of one or more of paclitaxel, docetaxel, carbazitaxel, larotaxel, ortataxel, tesetaxel or milataxel. In specific embodiments, R₁-R₉ groups of formula FX3 are those of one or more of paclitaxel, docetaxel, carbazitaxel, larotaxel, ortataxel, tesetaxel or milataxel. In specific embodiments, the cytotoxic taxane is paclitaxel, docetaxel, carbazitaxel, larotaxel, ortataxel, tesetaxel or milataxel.

In embodiments, a cytotoxic taxane has a taxane drug core of formula (FX5a):

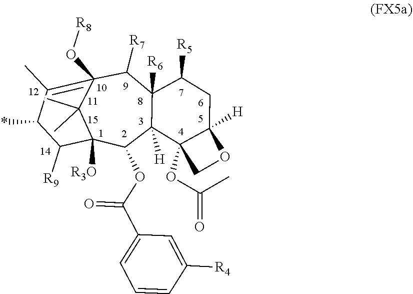

or salts thereof, where R₁-R₉ are as defined for formula FX5.

In embodiments, a cytotoxic taxane moiety that is derivatized at the 2′ side chain position with an α,ω-dicarboxylic acid has the following formula (FX5b):

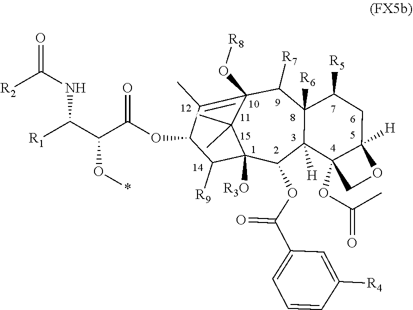

or salts thereof, where R1-R9 are as defined for formula FX5.

In some embodiments of any of the aspects and embodiments described herein, β-CyD can be any suitable water-soluble beta-cyclodextrin. In some embodiments, the beta-cyclodextrin is a derivatized beta-cyclodextrin. In some such embodiments, the derivatization comprises a modification of one or all of the hydroxyl groups of at least 1, or at least 3, or at least 5, or each of the 7 glucose subunits of the beta-cyclodextrin. In some embodiments, the derivatization comprises a modification of the hydroxyl groups of the hydrophobic cavity of the beta-cyclodextrin. In some embodiments, the derivatization comprises a modification of the hydroxyl groups of the hydrophilic outer surface of the beta cyclodextrin. Thus, in some embodiments, the degree of derivatization is a partial derivatization of cyclodextrin hydroxyl groups. In other embodiments, the degree of derivatization is a full derivatization of cyclodextrin hydroxyl groups.

In some embodiments wherein the β-CyD is derivatized, the derivatization comprises alkylation, such as methylation, propylation, or isopropylation, or acylation, such as acetylation. In some such embodiments, the derivatized β-CyD is an alkylated β-CyD. In some further such embodiments, the derivatized β-CyD is a methylated β-CyD.

In some embodiments of any of the aforementioned embodiments, β-CyD is a β-CyD selected from the group consisting of: heptakis beta-cyclodextrin, hetakis beta-cyclodextrin, randomly methylated beta-cyclodextrin, or 2-hydroxypropyl-beta cyclodextrin. In some further embodiments, β-CyD is heptakis beta-cyclodextrin.

In some embodiments of any of the aspects and embodiments described herein, A¹—X¹ can have any suitable structure, so long as those combinations result in stable chemical structures that would be suitable for pharmaceutical use. In some such embodiments, —X²—X¹—A¹ is —C(═O)—(CH₂)₈—CH₃, —C(═O)—(CH₂)₁₂—CH₃, —C(═O)—(CH₂)₁₆—CH₃, or —C(═O)—(CH₂)_{22—}CH₃. In some other such embodiments, —X²—X¹—A¹ is —C(═O)—(CH₂)₈—C(═O)—OH, —C(═O)—(CH₂)₁₂—C(═O)—OH, —C(═O)—(CH₂)₁₆—C(═O)—OH, or —C(═O)—(CH₂)₂₂—C(═O)—OH.

The selection of —X²—X¹—A¹ can depend on the nature of the connection to the drug moiety. For example, in embodiments where the —X²—X¹—A¹ connects to an oxygen atom or an NH group on the cytotoxic taxane drug moiety, then —X²—X¹—A¹ is selected from the group consisting of: —C(═O)—(CH₂)ₙ₁—C(═O)—OH; —C(═O)—(CH₂)ₙ₁—C(═O)—OCH₃; —C(═O)—(CH₂)ₙ₁—CH₃; —C(═O)—(C₁₋₆ alkylene)—C(═O)—O—(CH₂)ₙ₂—C(═O)—OH; —C(═O)—(C₁₋₆ alkylene)—NH—C(═O)—(CH₂)ₙ₁—C(═O)—OH; —C(═O)—(C₁₋₆ alkylene)—C(═O)—O—[(CH₂)₂—O—]ₙ₃(CH₂)ₙ₂—C(═O)—OH; —C(═O)—O—(CH₂)ₙ₂—C(═O)—OH; and —C(═O)—NH—(CH₂)ₙ₂—C(═O)—OH; wherein n1 is an integer from 8 to 24 (e.g., 8 to 24, 8 to 22, 8 to 20, 16 to 24, 16 to 22, 16 to 20), n2 is an integer from 13 to 25, and n3 is an integer from 1 to 25. In some further such embodiments, —X²—X¹—A¹ is selected from the group consisting of: —C(═O)—(CH₂)ₙ₁—C(═O)—OH; —C(═O)—(CH₂)ₙ₁—C(═O)—OCH₃; —C(═O)—(C₁₋₆ alkylene)—C(═O)—O—(CH₂)ₙ₂—C(═O)—OH; —C(═O)—(C₁₋₆ alkylene)—NH—C(═O)—(CH₂)ₙ₁—C(═O)—OH; —C(═O)—(C₁₋₆ alkylene)—C(═O)—O—[(CH₂)₂—O—]ₙ₃(CH₂)ₙ₂—C(═O)—OH; —C(═O)—O—(CH₂)ₙ₂—C(═O)—OH; and —C(═O)—NH—(CH₂)ₙ₂—C(═O)—OH. In some further such embodiments, —X²—X¹—A¹ is selected from the group consisting of: —C(═O)—(CH₂)ₙ₁—C(═O)—OH; —C(═O)—O—(CH₂)ₙ₂—C(═O)—OH; and —C(═O)—NH—(CH₂)ₙ₂—C(═O)—OH. In some other embodiments, —X²—X¹—A¹ is —C(═O)—(C₁₋₆ alkylene)—O—C(═O)—(CH₂)ₙ₁—C(═O)—OH, where n1 is an integer from 8 to 24 (e.g., 8 to 24, 8 to 22, 8 to 20, 10 to 24, 10 to 22, 10 to 20). In some embodiments of any of the aforementioned embodiments, n1 is an integer from 8 to 22, or from 16 to 22. In some embodiments of any of the aforementioned embodiments, n2 is an integer from 13 to 23, or from 17 to 21. In some embodiments of any of the aforementioned embodiments, n3 is an integer from 1 to 15, or from 1 to 10, or from 1 to 8. In some such embodiments, —X2—X1—A1 is —C(═O)—(C₁₋₆ alkylene)—C(═O)—O—(CH2)ₙ₃—OH, where n3 is an integer from 14 to 26, or an integer from 16 to 24, or an integer from 18 to 22.

In embodiments where the —X²—X¹—A¹ connects to an>N group on the cytotoxic taxane drug moiety, then —X²—X¹—A¹ is selected from the group consisting of: —CH₂—O—C(═O)—(CH₂)ₙ₁—C(═O)—OH; —CH₂—O—C(═O)—(CH₂)ₙ₁—C(═O)—OCH₃; —CH₂—O—C(═O)—(CH₂)ₙ₁—CH₃; —CH₂—O—C(═O)—(C₁₋₆ alkylene)—C(═O)—O—(CH₂)ₙ₂—C(═O)—OH; —CH₂—O—C(═O)—(C₁₋₆ alkylene)-NH—C(═O)—(CH₂)ₙ₁—C(═O)—OH; —CH₂—O—C(═O)—(C₁₋₆ alkylene)—C(═O)—O—[(CH₂)2—O—]ₙ₃(CH₂)ₙ₂—C(═O)—OH; —CH₂—O—C(═O)—O—(CH₂)ₙ₂—C(═O)—OH; and —CH₂—O—C(═O)—NH—(CH₂)ₙ₂—C(═O)—OH; wherein n1 is an integer from 8 to 24 (e.g., 8 to 24, 8 to 22, 8 to 20, 10 to 24, 10 to 22, 10 to 20), n2 is an integer from 13 to 25, and n3 is an integer from 1 to 25. In some further such embodiments, —X²—X¹—A¹ is selected from the group consisting of: —CH₂—O—C(═O)—(CH₂)ₙ₁—C(═O)—OH; —CH₂—O—C(═O)—(CH₂)ₙ₁—C(═O)—OCH₃; —CH₂—O—C(═O)—(C₁₋₆ alkylene)—C(═O)—O—(CH₂)ₙ₂—C(═O)—OH; —CH₂—O—C(═O)—(C₁₋₆ alkylene)—NH—C(═O)—(CH₂)ₙ₁—C(═O)—OH; —CH₂—O—C(═O)—(C₁₋₆ alkylene)—C(═O)—O—[(CH₂)₂—O—]ₙ₃(CH₂)ₙ₂—C(═O)—OH; —CH₂—O—C(═O)—O—(CH₂)ₙ₂—C(═O)—OH; and —C(═O)—NH—(CH₂)ₙ₂—C(═O)—OH. In some further such embodiments, —X²—X¹—A¹ is selected from the group consisting of: —CH₂—O—C(═O)—(CH₂)ₙ₁—C(═O)—OH; —CH₂—O—C(═O)—O—(CH₂)ₙ₂—C(═O)—OH; and —CH₂—O—C(═O)—NH—(CH₂)ₙ₂—C(═O)—OH. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹—A¹ is —CH₂—O—C(═O)—(C₁₋₆ alkylene)—C(═O)—O—(CH₂)ₙ₃—OH, where n3 is an integer from 14 to 26, or an integer from 16 to 24, or an integer from 18 to 22.

In embodiments where the —X²—X¹—A¹ connects to a —C(═O) group on the drug moiety, then —X²—X¹—A¹ is selected from the group consisting of: —O—(CH₂)ₙ₂—C(═O)—OH; —NH—(CH₂)ₙ₂—C(═O)—OH; —NH—(C₁₋₆ alkylene)—O—C(═O)—(CH₂)ₙ₁—C(═O)—OH; —O—(C₁₋₆ alkylene)—O—C(═O)—(CH₂)ₙ₁—C(═O)—OH; —NH—(C₁₋₆ alkylene)—O—C(═O)—(CH₂)ₙ₁—C(═O)—OCH₃; —O—(C₁₋₆ alkylene)—O—C(═O)—(CH₂)ₙ₁—C(═O)—OCH₃; —NH—(C₁₋₆ alkylene)—O—C(═O)—(CH₂)ₙ₁—CH₃; —O—(C₁₋₆ alkylene)—O—C(═O)—(CH₂)ₙ₁—CH₃; —NH—(C₁₋₆ alkylene)—C(═O)—O—[(CH₂)2—O—]ₙ₃(CH₂)ₙ₂—C(═O)—OH; and —O—(C₁₋₆ alkylene)—C(═O)—O—[(CH₂)2—O—]ₙ₃(CH₂)ₙ₂—C(═O)—OH; wherein n1 is an integer 12 to 24 (e.g., 12 to 24, 12 to 22, 12 to 20, 16 to 24, 16 to 22), n2 is an integer from 4 to 12, and n3 is an integer from 1 to 25. In some further such embodiments, —X²—X¹—A¹ is selected from the group consisting of: —O—(CH₂)ₙ₂—C(═O)—OH; —NH—(CH₂)ₙ₂—C(═O)—OH; —NH—(C₁₋₆ alkylene)—O—C(═O)—(CH₂)ₙ₁—C(═O)—OH; —O—(C₁₋₆ alkylene)—O—C(═O)—(CH₂)ₙ₁—C(═O)—OH; —NH—(C₁₋₆ alkylene)—O—C(═O)—(CH₂)ₙ₁—C(═O)—OCH₃; and —O—(C₁₋₆ alkylene)—O—C(═O)—(CH₂)ₙ₁—C(═O)—OCH₃. In some further such embodiments, —X²—X¹—A¹ is selected from the group consisting of: —O—(CH₂)ₙ₂—C(═O)—OH; —NH—(CH₂)ₙ₂—C(═O)—OH; —NH—(C₁₋₆ alkylene)—O—C(═O)—(CH₂)ₙ₁—C(═O)—OH; and —O—(C₁₋₆alkylene)—O—C(═O)—(CH₂)ₙ₁—C(═O)—OH. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹—A¹ is —O—(CH₂)ₙ₃—OH, where n3 is an integer from 14 to 26, or an integer from 16 to 24, or an integer from 18 to 22.

In embodiments where the —X²—X¹—A¹ connects to a C═* group on the drug moiety, then —X²—X¹—A¹ is selected from the group consisting of: ═N—O—(CH₂)ₙ₂—C(═O)—OH; ═N—NH—(CH₂)ₙ₂—C(═O)—OH; ═N—O—(CH₂)ₙ₂—C(═O)—OCH₃; ═N—NH—(CH₂)ₙ₂—C(═O)—OCH₃; ═N—O—(CH₂)ₙ₂—CH₃; ═N—NH—(CH₂)ₙ₂—CH₃; ═N—O—[(CH₂)2—O—]ₙ₃(CH₂)ₙ₂—C(═O)—OH; and ═N—NH—[(CH₂)2—O—]ₙ₃(CH₂)ₙ₂—C(═O)—OH; n2 is an integer from 13 to 25, and n3 is an integer from 1 to 25. In some further such embodiments, —X²—X¹—A¹ is selected from the group consisting of: ═N—O—(CH₂)ₙ₂—C(═O)—OH; ═N—NH—(CH₂)ₙ₂—C(═O)—OH; ═N—O—(CH₂)ₙ₂—C(═O)—OCH₃; and ═N—NH—(CH₂)ₙ₂—C(═O)—OCH₃. In some further such embodiments, —X²—X¹—A¹ is selected from the group consisting of: ═N—O—(CH₂)ₙ₂—C(═O)—OH and ═N—NH—(CH₂)ₙ₂—C(═O)—OH. In some embodiments of any of the aforementioned embodiments, n2 is an integer from 9 to 23, or from 17 to 21. In some embodiments of any of the aforementioned embodiments, n3 is an integer from 1 to 15, or from 1 to 10, or from 1 to 6. In some such embodiments, —X2—X1—A1 is selected from the group consisting of: ═N—O—(CH₂)ₙ₃—OH and ═N—NH—(CH₂)ₙ₃—OH, where n3 is an integer from 14 to 26, or an integer from 16 to 24, or an integer from 18 to 22.

The compounds described in any of the above embodiments can also exist as pharmaceutically acceptable salts. The term “pharmaceutically acceptable salts” refers to salts of the compounds which are not biologically or otherwise undesirable and are generally prepared by reacting the free base with a suitable organic or inorganic acid or by reacting the acid with a suitable organic or inorganic base. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, there can be formed an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate, and the like.

The compounds above can be made by standard organic synthetic methods, such as those illustrated in: Wuts et al., Greene's Protective Groups in Organic Synthesis (4th ed., 2006); Larock, Comprehensive Organic Transformations (2nd ed., 1999); and Smith et al., March's Advanced Organic Chemistry: Reactions, Mechanisms, and Structure (6th ed., 2007). Specific non-limiting examples are shown below in the Examples.

The compounds of the foregoing embodiments, including their pharmaceutically acceptable salts, are useful as cytotoxic agents or prodrugs thereof, and are therefore useful as compounds for the treatment of cancer.

In certain embodiments of any of the foregoing embodiments, the pharmaceutical composition also includes a carrier, such as an aqueous carrier. In some embodiments, the carrier includes sterile water. For example, in some such embodiments, sterile water makes up at least 50% by volume, or at least 60% by volume, or at least 70% by volume, or at least 80% by volume, at least 90% by volume, or 100% by volume based on the total volume of liquid materials in the pharmaceutical composition. The carrier can also include other liquid ingredients, such as phosphate buffered saline, tris buffered saline, saline solution, pharmaceutically acceptable saline, or other similar liquid ingredients commonly included in aqueous pharmaceutical formulations for parenteral administration. In preferred embodiments, the aqueous carrier does not include an organic solvent, such as ethanol, dimethyl sulfoxide, or ethylene glycol.

The cytotoxic taxane drug moiety of formulas (FX1), (FX1a), (FX2), (FX5), (FX5a), and (FX5b) can have any suitable molar ratio to the water-soluble beta-cyclodextrin (β-CyD) in the formulation. For example, in some embodiments of any of the foregoing embodiments, the molar ratio of the T² of formula (FX1) or (FX1a) to the β-CyD ranges from 1:1 to 1:10, or from 1:1 to 1:7, or from 1:1 to 1:5. For example, in some embodiments of any of the foregoing embodiments, the molar ratio of the cytotoxic taxane drug moiety of formulas (FX2) and (FX5)-(FX5b) to the β-CyD ranges from 1:1 to 1:10, or from 1:1 to 1:7, or from 1:1 to 1:5. In some embodiments, the molar ratio of the T² of formulas (FX1) and (FX1a) to the β-CyD ranges from 1:2 to 3:10. In some embodiments, the molar ratio of the cytotoxic taxane drug moiety of formulas (FX2) and (FX5)-(FX5b) to the β-CyD ranges from 1:2 to 3:10. In some embodiments of any of the foregoing embodiments, the molar ratio of the cytotoxic taxane drug moiety of formulas (FX1), (FX1a), (FX2), (FX5), (FX5a), and (FX5b) to the β-CyD is about 1:5, or is about 1:4, or is about 1:3, or is about 1:2, or is about 1:2.3, or is about 3:7 wherein the term “about,” in this instance means 1:±0.5, such that “about 1:5” refers to a range from 1:4.5 to 1:5.5.

In general, the pharmaceutical compositions used herein include: a compound, which comprises a cytotoxic taxane drug moiety, a protein binding moiety, a water-soluble beta-cyclodextrin, and a carrier, which comprises water.

In some embodiments, the protein binding moiety is capable of binding to an endogenous carrier protein, such as a human serum albumin (HSA) protein or a mimetic thereof, i.e., a protein whose sequence is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA.

In certain embodiments, the compounds bind non-covalently to HSA in a subject. In some embodiments, the compound and the HSA are non-covalently associated with each other with a binding constant (K_{b}) of at least 10² M⁻¹, or at least 10³ M⁻¹, or at least 10⁴ M⁻¹, or at least 10⁵ M⁻¹ at 25° C.

In other embodiments, the inclusion complex or salt thereof, or the aqueous pharmaceutically acceptable solution is pre-mixed with HSA. Thus, in these embodiments, the protein binding moiety is capable of binding with HSA, or a mimetic thereof (i.e., a protein whose sequence is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA), prior to administration to a subject. In some embodiments, pre-mixing the compounds with HSA is useful for experimental studies in non-human animal models. In some such embodiments, the molar ratio of cytotoxic taxane drug moiety to HSA ranges from 1:1 to 40:1. In further embodiments, the molar ratio of cytotoxic taxane drug moiety to HSA is 5:1. In preferred embodiments, however, the compounds bind to endogenous HSA in a subject. In some embodiments of any of the foregoing embodiments, the molar ratio of cytotoxic taxane drug moiety to HSA is about 30:1, or is about 10:1, or is about 8:1, or is about 6:1, or is about 5:1, or is about 4:1, wherein the term “about,” in this instance means ±0.5:1, such that “about 5:1” refers to a range from 4.5:1 to 5.5:1.

The pharmaceutical compositions of any of the foregoing aspects and embodiments can also include certain additional ingredients, such as those commonly employed in pharmaceutical compositions for parenteral administration, such as excipients commonly used in fluids suitable for intravenous administration. Non-limiting examples of additional components include water, various salts (such as sodium chloride), various sugars (such as glucose, dextrose, and the like), and pH buffers.

In some embodiments, the pharmaceutical compositions do not include thickeners (such as cellulosic materials), amino acids, proteins (such as albumin), separately added liposomes, polymers, organic solvents (such as ethanol, ethylene glycol, dimethyl sulfoxide, and the like), preservatives, surfactants, or emulsifiers, and the like.

Such pharmaceutical compositions may be administered according to any suitable dosage regimen and in any suitable amount. For example, when administered by intravenous infusion, the compound of formula (FX1), or any other formula described herein, is administered as a dose of 10 to 500 mg/kg with respect to the cytotoxic taxane drug moiety over the course of 1 to 48 hours. In some further embodiments, the compound of formula (FX1), or any other formula described herein, is administered as a dose of 5 to 250 mg/kg with respect to the cytotoxic taxane drug moiety over the course of 1 to 24 hours. Such infusions can be part of a multi-dose regimen. For example, following an initial administration at the quantities set forth above, the subject is administered a similar dose on one, two, three, four, five, six, or more additional occasions. Such additional administrations may be separated from each other for any suitable time period, such as one week, two weeks, three weeks, four weeks, five weeks, or six weeks.

In the methods and uses disclosed herein, the foregoing compounds and compositions are administered to a subject for the treatment of cancer and related disorders. In some embodiments, these compounds and compositions are be used for administration to a subject who has or has had a cancerous tumor. In some embodiments, the cancer is a cancer that comprises cells that overexpress fatty acid uptake proteins. In some further embodiments, the cancer is a cancer that comprises cells that overexpress CD36. As used herein, the term “overexpress” refers to an abnormally high expression of a particular protein or class of proteins relative to healthy cells of a similar type. For example, in the case of certain breast cancers, the cells of the tumor may be said to overexpress certain proteins because the cancerous cells express certain proteins to a greater degree than the cells of normal, healthy breast tissue. The overexpression can be of any suitable degree. For example, in some embodiments, the cancer comprises cells that overexpress fatty acid uptake proteins (or, in some embodiments, CD36) at a concentration of at least 10% more, or at least 25% more, or at least 50% more, or at least 100% more than the cells of normal, healthy tissue of the infected tissue type.

Such tumors can affect various systems of the body. Thus, the cancers being treated in some embodiments include, but are not limited to: cancers of epithelial origin, such as breast cancer, prostate cancer, ovarian cancer, and colon cancer; hepatic carcinomas and gliomas; gastric cancer; glioblastomas; oral carcinomas, such as oral squamous cell carcinoma; acute myeloid leukemia; lung squamous cell carcinomas; and bladder cancer. Various cancers in which CD36 expression plays a role are set forth in Enciu et al., BIOMED. RES. INT., vol. 2018:7801202 (published online Jul. 4, 2018), the contents of which are hereby incorporated by reference.

In some embodiments, the cancer is a metastatic cancer, such as a metastatic tumor. In some further embodiments, the cancer is metastatic breast cancer. In some other embodiments, the cancer is metastatic prostate cancer. In some other embodiments, the cancer is metastatic ovarian cancer. In some other embodiments, the cancer is metastatic colon cancer. In some other embodiments, the cancer is metastatic hepatic cancer, such as a metastatic hepatic carcinoma or glioma. In some embodiments, the cancer is breast cancer, ovarian cancer, lung cancer, Kaposi's sarcoma, fibrosarcoma, prostate cancer, or pancreatic cancer.

In some embodiments, the compounds of formula (FX1) and (FX1a) are administered in combination with one or more other compounds. In some embodiments, the one or more other compounds includes another cytotoxin, such as a small-molecule cytotoxin. In some other embodiments, the one or more other compounds comprises one or more oligonucleotide compounds.

The administration of compounds of formula (FX1) and (FX1a), or any other formula described herein, can also be included within a dosage regimen that includes administering a therapeutically effective amount of one or more additional chemotherapeutic agents to the subject in need thereof. Non-limiting examples of such additional chemotherapeutic agents include, but are not limited to taxanes, topoisomerase I inhibitors, topoisomerase II inhibitors, alkylating agents, anthracycline compounds, platinum-based compounds, anti-folate compounds, purine analogs, vinca alkaloids, kinase inhibitors, ubiquitin ligase modulators, androgen receptor agonists, proteasome inhibitors, Hedgehog signaling pathway modulators, epothilone compounds, cytotoxic oligonucleotides, cyctotoxic proteins, and the like. In some embodiments, the one or more additional chemotherapeutic agents comprises cisplatin, carboplatin, cyclophosphamide, vinorelbine, topotecan, doxorubicin, or gemcitabine.

In certain embodiments, the compounds of formula (FX1) or (FX1a), or any other formula described herein, are used to “prime” the cancer and improve the potential efficacy of immunomodulating agents. Thus, in some embodiments, the compounds of formula (FX1) or (FX1a), or any other formula described herein, are administered in combination with one or more immunomodulating agents. Any suitable immunomodulating agents can be used, including, but not limited to: monoclonal antibodies (such as alemtuzumab, atezolizumab, ipilimumab, nivolumab, pertuzumab, trastzumab, and pembrolizumab), anti-CD47 antibodies, anti-SIRP-alpha antibodies, anti-GD2 antibodies, anti-PD-1 antibodies, anti-PD-L1 antibodies, immune checkpoint inhibitors (such as CTLA-4 inhibitors), and toll-like receptor (TLR) agonists Thus, the administration of a compound of the formula can, in some embodiments, be part of a dosage regimen that includes administering one or more immunomodulating agents. For example, in certain embodiments, the subject is treated initially with a dosage (or dosage regimen) of a compound of formula (FX1) or (FX1a), or any other formula described herein, followed by a subsequent administration with one or more immunomodulating compounds.

In certain embodiments, the administration of compounds of formula (FX1) or (FX1a), or any other formula described herein, can also be included within a dosage regimen that includes administering a therapeutically effective amount of one or more cancer supportive drugs to the subject. Non-limiting examples of such cancer supportive drugs include pamidronate, allopurinol, rasburicase, amifostine, dexrazoxane, or a colony-stimulating factor.

The foregoing compounds may be formulated into pharmaceutical compositions in any suitable manner. In general, as compounds for the treatment of cancer, such pharmaceutical formulations are aqueous formulations suitable for parenteral administration, such as intravenous or intra-arterial administration.

### Aspects of the Invention

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect A13) that references an aspect (e.g., Aspect A1) for which there are sub-aspects having the same top level number (e.g., Aspect A1a, A1b, A1c, and so forth) necessarily includes reference to those sub-aspects A1a, A1b, A1c, and so forth. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1-B12, or any preceding aspect, . . . ” means that any aspect prior to aspect B13 is referenced, including aspects B1-B12 and all of the “A” aspects). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y, even though “less than Y” in isolation does not include Y.

Aspect A1: An inclusion complex characterized by formula (FX1):

(A¹—X¹—X²—T²)m:(β-CyD)n (FX1)
or a salt thereof,
wherein:
A¹ is a carboxylic acid group, a carboxylate anion, or a carboxylate ester;
T² is a cytotoxic taxane drug moiety, which has a molecular weight of no more than 1600 Da; for example, the cytotoxic taxane drug moiety has a molecular weight of no more than 1600 Da, no more than 1400 Da, no more than 1200 Da, no more than 1000 Da.; or no more than 900 Da;
X¹ is an optionally substituted alkylene group having 8-22 carbon atoms (e.g., 8-22 carbon atoms, 10-22 carbon atoms, 12-22 carbon atoms, 16-22 carbon atoms, 8-20 carbon atoms, 10-20 carbon atoms, 12-20 carbon atoms, or 16-20 atoms);
X2 is a direct bond, an organic group moiety, —O—C(═O), —O—C(═O)—O—, —C(═O)—, —O—, —S—, —S(═O)—, —S(═O)₂—, —S—S—, —N═, ═N—, —N(H)—, —N═N—N(H)—, —N(H)—N═N—, —N(OH)—, or —N(═O)—;
β-CyD is a water-soluble beta-cyclodextrin;
m is the number of derivatized cytotoxic taxanes associated with the beta-cyclodextrin in the inclusion complex; and
n is the number of beta-cyclodextrin associated with the derivatized cytotoxic taxane drug moiety in the inclusion complex;
wherein the ratio of m:n ranges from 1:2 to 3:10 (e.g., 3:7).

Aspect A2: The inclusion complex of aspect A1, wherein T² is a paclitaxel moiety, a docetaxel moiety, a cabazitaxel moiety, a larotaxel moiety, an ortataxel moiety, a tesetaxel moiety, or a milataxel moiety.

Aspect A3: The inclusion complex of aspect A1 or A2, wherein X¹ is an unsubstituted alkylene group having 8-22 carbon atoms (e.g., 8-22 carbon atoms, 10-22 carbon atoms, 12-22 carbon atoms, 16-22 carbon atoms, 8-20 carbon atoms, 10-20 carbon atoms, 12-20 carbon atoms, or 16-20 atoms).

Aspect A4: The inclusion complex of any one of aspects A1-A3, wherein X¹ is an unsubstituted alkylene group having 14-18 carbon atoms (e.g., 14, 15, 16, 17, or 18 carbon atoms).

Aspect A5: The inclusion complex of any one of aspects A1-A4, wherein X¹ is an unsubstituted alkylene group having 16 carbon atoms.

Aspect A6: The inclusion complex of any one of aspects A1-A5, wherein X² is —O—C(═O), —O—C(═O)—O—, —C(═O)—, or —O—.

Aspect A7: The inclusion complex of any one of aspects A1-A6, wherein β-CyD is a derivatized β-CyD.

Aspect A8: The inclusion complex of aspect A7, or any preceding aspect, wherein the derivatized β-CyD is an alkylated β-CyD or an acylated β-CyD.

Aspect A8a: The inclusion complex of aspect A7 or A8, or any preceding aspect, wherein the derivatized β-CyD is an alkylated β-CyD.

Aspect A8b: The inclusion complex of aspect A7 or A8, or any preceding aspect, wherein the derivatized β-CyD is an acylated β-CyD.

Aspect A9: The inclusion complex of aspect A7, or any preceding aspect, wherein the derivatized β-CyD comprises a methylated β-CyD, a propylated β-CyD, or an isopropylated β-CyD.

Aspect A9a: The inclusion complex of aspect A9, or any preceding aspect, wherein the derivatized β-CyD comprises a methylated β-CyD.

Aspect A10: The inclusion complex of any one of aspects A1-A9a, wherein β-CyD is a heptakis beta-cyclodextrin, a hetakis beta-cyclodextrin, a randomly methylated beta-cyclodextrin, or a 2-hydroxypropyl-beta cyclodextrin.

Aspect A11: The inclusion complex of any one of aspects A1-A10, wherein β-CyD is a heptakis beta-cyclodextrin.

Aspect A12: The inclusion complex of any one of aspects A1-A11, wherein the molar ratio of T² to β-CyD is between 1:1 and 1:10 (e.g., between 1:1 and 1:10, between 1:1 and 1:7, between 1:1 and 1:5, or between 1:1 and 1:3).

Aspect A13: The inclusion complex of any one of aspects A1-A12, wherein the molar ratio of T² to β-CyD is between 1:1 and 1:7 (e.g., between 1:1 and 1:7, between 1:1 and 1:5, or between 1:1 and 1:3).

Aspect A14: The inclusion complex of any one of aspects A1-A13, wherein the molar ratio of T² to β-CyD is between 1:1 and 1:5 (e.g., between 1:1 and 1:5 or between 1:1 and 1:3).

Aspect A15: The inclusion complex of any one of aspects A1-A14, wherein the molar ratio of T² to β-CyD is between 1:1 and 1:3.

Aspect A16: The inclusion complex of any one of aspects A1-A15, wherein the molar ratio of T² to β-CyD is 1:2.3.

Aspect A17: The inclusion complex of any one of aspects A1-A16, wherein T² is a paclitaxel moiety, wherein the paclitaxel moiety is characterized by formula (FX2):

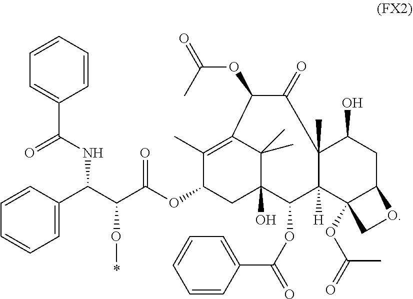 [SMILES: *O[C@@H](C(=O)O[C@H]1C[C@@]2(O)[C@@H](OC(=O)c3ccccc3)[C@H]3[C@](C)(CC(=O)[C@H](OC(C)=O)C(=C1C)C2(C)C)[C@@H](O)C[C@H]1OC[C@]13OC(C)=O)[C@@H](NC(=O)c1ccccc1)c1ccccc1]

Aspect B1: An aqueous pharmaceutically acceptable solution comprising:
an aqueous carrier; and
one or more than one inclusion complex or salt thereof of any one of aspects A1-A17, wherein the total concentration of the cytotoxic taxane drug moiety in the aqueous solution is equal to 0.5 mg/mL or higher (e.g., 0.5 mg/mL or higher, 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect B1a: An aqueous pharmaceutically acceptable solution comprising:
an aqueous carrier; and
the inclusion complex or salt thereof of any one of aspects A1-A17, wherein the total concentration of the cytotoxic taxane drug moiety in the aqueous solution is equal to 0.5 mg/mL or higher (e.g., 0.5 mg/mL or higher, 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect B1b: An aqueous pharmaceutically acceptable solution comprising:
an aqueous carrier; and
at least two inclusion complexes (e.g., two distinct inclusion complexes, three distinct inclusion complexes, four distinct inclusion complexes, five distinct inclusion complexes, etc.) or salt thereof of any one of aspects A1-A17, wherein the total concentration of the cytotoxic taxane drug moiety in the aqueous solution is equal to 0.5 mg/mL or higher (e.g., 0.5 mg/mL or higher, 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect B2: The aqueous pharmaceutically acceptable solution of any one of aspects B1-B1b, or any preceding aspect, wherein the total concentration of the cytotoxic taxane drug moiety in the pharmaceutically acceptable aqueous solution is equal to 1 mg/mL or higher (e.g., 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect B3: The aqueous pharmaceutically acceptable solution of any one of aspects B1-B2, or any preceding aspect, wherein the total concentration of the cytotoxic taxane drug moiety in the aqueous pharmaceutically acceptable solution is equal to 5 mg/mL or higher (e.g., 5 mg/mL or higher, or 7.5 mg/mL or higher; optionally less than 15 mg/mL, less than 12 mg/mL, or less than 11 mg/mL).

Aspect B4: The aqueous pharmaceutically acceptable solution of aspect B1, or any preceding aspect, wherein the total concentration of the cytotoxic taxane drug moiety in the aqueous pharmaceutically acceptable solution is equal to 10.91 mg/mL.

Aspect B4a: The aqueous pharmaceutically acceptable solution of aspect B1, or any preceding aspect, wherein the total concentration of the cytotoxic taxane drug moiety in the aqueous pharmaceutically acceptable solution is equal to 10 mg/mL.

Aspect B4b: The aqueous pharmaceutically acceptable solution of aspect B1, or any preceding aspect, wherein the total concentration of the cytotoxic taxane drug moiety in the aqueous pharmaceutically acceptable solution is equal to 11 mg/mL.

Aspect B5: The aqueous pharmaceutically acceptable solution of any one of aspects B1-B4b, or any preceding aspect, wherein the aqueous pharmaceutically acceptable solution does not include albumin.

Aspect B6: The aqueous pharmaceutically acceptable solution of any one of aspects B1-B5, or any preceding aspect, wherein the aqueous pharmaceutically acceptable solution does not include proteins.

Aspect B7: The aqueous pharmaceutically acceptable solution of any one of aspects B1-B6, or any preceding aspect, wherein the aqueous pharmaceutically acceptable solution does not include liposomes.

Aspect B8: The aqueous pharmaceutically acceptable solution of any one of aspects B1-B7, or any preceding aspect, wherein the aqueous pharmaceutically acceptable solution does not include polymers.

Aspect B9: The aqueous pharmaceutically acceptable solution of any one of aspects B1-B8, or any preceding aspect, wherein the aqueous pharmaceutically acceptable solution does not include surfactants.

Aspect B10: The aqueous pharmaceutically acceptable solution of any one of aspects B1-B9, or any preceding aspect, wherein the aqueous pharmaceutically acceptable solution does not include dimethyl sulfoxide.

Aspect B11: The aqueous pharmaceutically acceptable solution of any one of aspects B1-10, or any preceding aspect, wherein the aqueous pharmaceutically acceptable solution does not include ethanol.

Aspect B12: The aqueous pharmaceutically acceptable solution of any one of aspects B1-B11, or any preceding aspect, wherein the aqueous pharmaceutically acceptable solution does not include ethylene glycol.

Aspect B13: The aqueous pharmaceutically acceptable solution of any one of aspects B1-B12, or any preceding aspect, wherein the inclusion complex is added to the aqueous carrier in the form of a lyophilized powder.

Aspect B14: The aqueous pharmaceutically acceptable solution of any one of aspects B1-B13, or any preceding aspect, wherein the aqueous carrier is sterile water for injection or pharmaceutically acceptable saline.

Aspect B15: The aqueous pharmaceutically acceptable solution of any one of aspects B1-14, or any preceding aspect, wherein the aqueous carrier does not comprise an organic solvent.

Aspect B16: The aqueous pharmaceutically acceptable solution of any one of aspects B1-B15, or any preceding aspect, further comprising one or more of an immunomodulating agent.

Aspect B17: The aqueous pharmaceutically acceptable solution of aspect B16, or any preceding aspect, wherein the immunomodulating agent comprises one or more of alemtuzumab, atezolizumab, ipilimumab, nivolumab, pembrolizumab, trastzumab, or pertuzumab.

Aspect B18: The aqueous pharmaceutically acceptable solution of any one of aspects B1-B17, comprising one inclusion complex or salt thereof of any one of aspects A1-A17, wherein the concentration of the cytotoxic taxane drug moiety in the aqueous solution is equal to 0.5 mg/mL or higher (e.g., 0.5 mg/mL or higher, 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect B19: The aqueous pharmaceutically acceptable solution of any one of aspects B1-B17, or any preceding aspect, comprising more than one inclusion complex (e.g., two distinct inclusion complexes, three distinct inclusion complexes, four distinct inclusion complexes, five distinct inclusion complexes, etc.) or salt thereof of any one of aspects A1-A17, wherein the total concentration of the cytotoxic taxane drug moiety in the aqueous solution is equal to 0.5 mg/mL or higher (e.g., 0.5 mg/mL or higher, 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect C1: An aqueous pharmaceutically acceptable solution comprising:
a water soluble beta-cyclodextrin; and
one or more derivatized cytotoxic taxane drug moiety of formula (FX1a):

A¹—X¹—X²—T² (FX1a)
or a salt thereof,
wherein:
A¹ is a carboxylic acid group, a carboxylate anion, or a carboxylate ester;
T² is a cytotoxic taxane drug moiety, which has a molecular weight of no more than 1600 Da for example, the cytotoxic taxane drug moiety has a molecular weight of no more than 1600 Da, no more than 1400 Da, no more than 1200 Da, no more than 1000 Da.; or no more than 900 Da;
X¹ is an optionally substituted alkylene group having 8-22 carbon atoms (e.g., 8-22 carbon atoms, 10-22 carbon atoms, 12-22 carbon atoms, 16-22 carbon atoms, 8-20 carbon atoms, 10-20 carbon atoms, 12-20 carbon atoms, or 16-20 atoms); and
X² is a direct bond, an organic group moiety, —O—C(═O), —O—C(═O)—O—, —C(═O)—, —O—, —S—, —S(═O)—, —S(═O)₂—, —S—S—, —N═, ═N—, —N(H)—, —N═N—N(H)—, —N(H)—N═N—, —N(OH)—, or —N(═O)—;
wherein the total concentration of the one or more derivatized cytotoxic taxane drug moiety in the aqueous pharmaceutically acceptable solution is equal to 0.5 mg/mL or higher (e.g., 0.5 mg/mL or higher, 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect C1a: An aqueous pharmaceutically acceptable solution comprising:
a water soluble beta-cyclodextrin; and
a derivatized cytotoxic taxane drug moiety of formula (FX1a):

A¹—X¹—X²—T² (FX1a)
or a salt thereof,
wherein:
A¹ is a carboxylic acid group, a carboxylate anion, or a carboxylate ester;
T² is a cytotoxic taxane drug moiety, which has a molecular weight of no more than 1600 Da for example, the cytotoxic taxane drug moiety has a molecular weight of no more than 1600 Da, no more than 1400 Da, no more than 1200 Da, no more than 1000 Da.; or no more than 900 Da;
X¹ is an optionally substituted alkylene group having 8-22 carbon atoms (e.g., 8-22 carbon atoms, 10-22 carbon atoms, 12-22 carbon atoms, 16-22 carbon atoms, 8-20 carbon atoms, 10-20 carbon atoms, 12-20 carbon atoms, or 16-20 atoms); and
X2 is a direct bond, an organic group moiety, —O—C(═O), —O—C(═O)—O—, —C(═O)—, —O—, —S—, —S(═O)—, —S(═O)₂—, —S—S—, —N═, ═N—, —N(H)—, —N═N—N(H)—, —N(H)—N═N—, —N(OH)—, or —N(═O)—;
wherein the total concentration of the derivatized cytotoxic taxane drug moiety in the aqueous pharmaceutically acceptable solution is equal to 0.5 mg/mL or higher (e.g., 0.5 mg/mL or higher, 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect C1b: An aqueous pharmaceutically acceptable solution comprising:
a water soluble beta-cyclodextrin; and
at least two derivatized cytotoxic taxane drug moieties (e.g., two distinct derivatized cytotoxic taxane drug moieties, three distinct derivatized cytotoxic taxane drug moieties, four distinct derivatized cytotoxic taxane drug moieties, five distinct derivatized cytotoxic taxane drug moieties, etc; optionally less than 20 distinct derivatized cytotoxic taxane drug moieties, optionally less than 15 distinct derivatized cytotoxic taxane drug moieties) of formula (FX1a):

A¹—X¹—X²—T² (FX1a)
or a salt thereof,
wherein:
A¹ is a carboxylic acid group, a carboxylate anion, or a carboxylate ester;
T² is a cytotoxic taxane drug moiety, which has a molecular weight of no more than 1600 Da for example, the cytotoxic taxane drug moiety has a molecular weight of no more than 1600 Da, no more than 1400 Da, no more than 1200 Da, no more than 1000 Da.; or no more than 900 Da;
X¹ is an optionally substituted alkylene group having 8-22 carbon atoms (e.g., 8-22 carbon atoms, 10-22 carbon atoms, 12-22 carbon atoms, 16-22 carbon atoms, 8-20 carbon atoms, 10-20 carbon atoms, 12-20 carbon atoms, or 16-20 atoms); and
X2 is a direct bond, an organic group moiety, —O—C(═O), —O—C(═O)—O—, —C(═O)—, —O—, —S—, —S(═O)—, —S(═O)₂—, —S—S—, —N═, ═N—, —N(H)—, —N═N—N(H)—, —N(H)—N═N—, —N(OH)—, or —N(═O)—;
wherein the total concentration of the at least two derivatized cytotoxic taxane drug moieties in the aqueous pharmaceutically acceptable solution is equal to 0.5 mg/mL or higher (e.g., 0.5 mg/mL or higher, 1 mg/mL or higher, 2.5 mg/mL or higher, 5 mg/mL or higher, or 7.5 mg/mL or higher; optionally less than 15 mg/mL, less than 12 mg/mL, or less than 11 mg/mL).

Aspect C2: The aqueous pharmaceutically acceptable solution of aspect C1, or any preceding aspect, wherein the molar ratio of derivatized cytotoxic taxane drug moiety to water soluble beta-cyclodextrin ranges from 1:1 to 3:10 (e.g., 3:7).

Aspect C3: The aqueous pharmaceutically acceptable solution of aspect C1 or C2, or any preceding aspect, wherein the derivatized cytotoxic taxane drug moiety and the water soluble beta-cyclodextrin are substantially in the form of an inclusion complex.

Aspect D1: A method of treating cancer, the method comprising administering to a subject a therapeutically effective amount of the one or more inclusion complex of any one of aspects A1-A17, or any preceding aspect, or the aqueous pharmaceutically acceptable solution of any one of aspects B1-C3, or any preceding aspect.

Aspect D2: The method of aspect D1, or any preceding aspect, where administration is by intravenous injection or infusion.

Aspect D3: The method of aspect D1 or D2, or any preceding aspect, wherein the cancer comprises cells that overexpress one or more fatty acid uptake proteins.

Aspect D4: The method of aspect D1 or D2, or any preceding aspect, wherein the cancer is breast cancer, ovarian cancer, lung cancer, Kaposi's sarcoma, fibrosarcoma, prostate cancer, or pancreatic cancer.

Aspect D5: The method of any one of aspects D1-D4, or any preceding aspect, wherein the subject is a neonate, an infant, a child, or an adolescent.

Aspect D6: The method of any one of aspects D1-D5, or any preceding aspect, wherein the one or more inclusion complex or the aqueous pharmaceutically acceptable solution is administered to the subject in combination with one or more additional therapeutic agents (e.g., one additional therapeutic agent, two additional therapeutic agents, three additional therapeutic agents, four additional therapeutic agents, five additional therapeutic agents, etc.),

Aspect D7: The method of any one of aspects D1-D6, or any preceding aspect, wherein the method of treating cancer is combined with one or more additional methods known to treat cancer (e.g., one additional method, two additional methods, three additional methods, etc).

Aspect D8: The method of aspect D7, or any preceding aspect, wherein the one or more additional methods known to treat cancer comprises administering to the subject a therapeutically effective amount of a chemotherapeutic agent.

Aspect D9: The method of aspect D8, or any preceding aspect, wherein the chemotherapeutic agent is cisplatin, carboplatin, cyclophosphamide, vinorelbine, topotecan, doxorubicin, or gemcitabine.

Aspect D10: The method of aspect D9, or any preceding aspect, wherein the one or more additional methods known to treat cancer comprises administering to the subject a therapeutically effective amount of a cancer supportive drug.

Aspect D11: The method of aspect D10, or any preceding aspect, wherein the cancer supportive drug is pamidronate, allopurinol, rasburicase, arnifostine, dexrazoxane, or a colony-stimulating factor.

Aspect D12: The method of any one of aspects D1-D11, or any preceding aspect, further comprising the step of: pre-mixing the one or more inclusion complex or the aqueous pharmaceutically acceptable solution with human serum albumin prior to the administering step.

Aspect D13: The method of aspect D12, or any preceding aspect, wherein the molar ratio of cytotoxic taxane drug moiety to human serum albumin ranges from 1:1 to 10:1 (e.g., 1:1. 3:1, 5:1, 6:1, 7:1, 8:1, 9:1, or 10:1).

Aspect D14: The method of claim 51 or 52, wherein the molar ratio of cytotoxic taxane drug moiety to human serum albumin is 5:1.

Aspect E1: A method for preparation of an improved pharmaceutically acceptable aqueous solution containing 9.5 mM or greater of a taxane moiety, which comprises the steps of:
a. preparing the inclusion complex of any one of aspects A1-A17, or any preceding aspect, by mixing A¹—X¹—X²—T² is with the β-CyD wherein the molar ratio of β-CyD to A¹—X¹—X²—T² is greater than 1 (e.g., a molar ratio of β-CyD to A¹—X¹—X²—T² is of 2:1, 2.3:1, 3:1, 7:3, 5:1, or 10:1) to form one or more inclusion complexes; and
b. dissolving the inclusion complex in a selected pharmaceutically acceptable aqueous solvent at a concentration equal to or greater than 9.5 mM (e.g., equal to or greater than 9.5 mM, equal to or greater than 10 mM, equal to or greater than 15 mM, equal to or greater than 20 mM; optionally equal to or less than 100 mM, or equal to or less than 75 mM) with respect to the taxane drug moiety.

Aspect F1: A method of generating the inclusion complex of any one of aspects A1-A17, or any preceding aspect, the method comprising the steps of:
a. providing a solvent;
b. dissolving A¹—X¹—X²—T² is in the solvent to form a solution;
c. preparing an aqueous β-CyD by dissolving β-CyD in water;
d. combing the aqueous β-CyD and the solution wherein the molar ratio of β-CyD to A¹—X¹—X²—T² is is greater than 1 to form a mixture; and
e. removing the solvent from the mixture;
wherein the removing step results in the formation of a solid, thereby generating the inclusion complex.

Aspect F2: The method of aspect F1, or any preceding aspect, further comprising the step of: stirring the mixture of step d. at room temperature for at least 12 hours (e.g., at least 12 hours, at least 13 hours, at least 14 hours, at least 15 hours, at least 16 hours; optionally less than 24 hours) before the combining step.

Aspect F3: The method of aspect F1 or F2, or any preceding aspect, further comprising the step of: filtering the mixture prior to the step of removing the solvent from the mixture.

Aspect F4: The method of any one of aspects F1-F3, or any preceding aspect, wherein the removing the solvent from the mixture comprises lyophilization.

Aspect F5: The method of any one of aspects F1-F4, or any preceding aspect, wherein the solvent comprises one or more of ethanol, methanol, tert-butanol, acetonitrile, or any acceptable polar, protic or aprotic, organic solvent.

Aspect F6: The method of any one of aspects F1-F5, or any preceding aspect, further comprising reconstituting the solid with a pharmaceutically acceptable aqueous carrier to form an aqueous solution at a concentration equal to or greater than 6.8 mM (equal to or greater than 6.8 mM, equal to or greater than 7 mM, equal to or greater than 8 mM, equal to or greater than 9.5 mM, equal to or greater than 10 mM, equal to or greater than 15 mM, etc.) with respect to the cytotoxic taxane drug moiety.

Aspect F7: The method of aspect F6, or any preceding aspect, wherein the pharmaceutically acceptable aqueous carrier is one or more of phosphate buffered saline, tris buffered saline, saline solution, sterile water for injection, or sterile saline for injection.

Aspect F7a: The method of aspect F7, or any preceding aspect, wherein the pharmaceutically acceptable aqueous carrier is sterile water for injection or sterile saline for inj ecti on.

Aspect F8: The method of aspect F7, or any preceding aspect, wherein the pharmaceutically acceptable aqueous carrier does not include an organic solvent.

### EXAMPLES

The invention can be further understood by the following non-limiting examples. The examples are provided to illustrate some of the concepts described within this disclosure. While each example is considered to provide specific individual embodiments of composition and methods of preparation and use, none of the examples should be considered to limit the more general embodiments described herein.

In the following examples, efforts have been made to ensure accuracy with respect to numbers used (e.g., amounts, temperature, etc.) but some experimental error and deviation should be accounted for.

Materials: The following descriptions of materials describe the materials used in one or more of the below Examples, as applicable. To the extent of any conflict between the materials of these descriptions and those provided in a particular Example, the Example controls.

All materials and reagents were purchased from Sigma Aldrich or Fisher Chemicals. ODDA was donated by Elevance Renewable Sciences, Inc. (Woodridge, Illinois). HPLC analyses of all materials were performed on a Jupiter 4 μ Proteo 90 Å Phenomenex column (150×4.60 mm) with a binary gradient, using a Hitachi-Elite LaChrom 2130 pump that was equipped with a Hitachi-Elite LaChrom L-2420 UV-Vis detector. Separation was achieved with a flow rate of 1 mL min-1 and the following mobile phase: 0.1% trifluoroacetic acid in H₂O (buffer A) and 0.1% trifluoroacetic acid in ACN (buffer B). All cell lines were obtained from an in-house sub-culture originally purchased from ATCC. Cells were incubated at 37° C. at 5% CO₂ using EMEM (high glucose, no glutamine, Life Technologies/Gibco, Cat. 11960044) supplemented with 10% FBS (heat inactivated, Omega) and antibiotics (Penicillin-Streptomycin, Life Technologies Cat 15140122). DMEM without phenol red (Life Technologies, Cat 31053028) was also used for the purpose of. Cell cultures were maintained by subculturing in flasks every 4-7 days when cells became confluent using trypsin-EDTA, 0.25% (Life Technologies, Cat 25200114).

### Example 1

This Example describes a method used to synthesize PTX-ODDA-CyD.

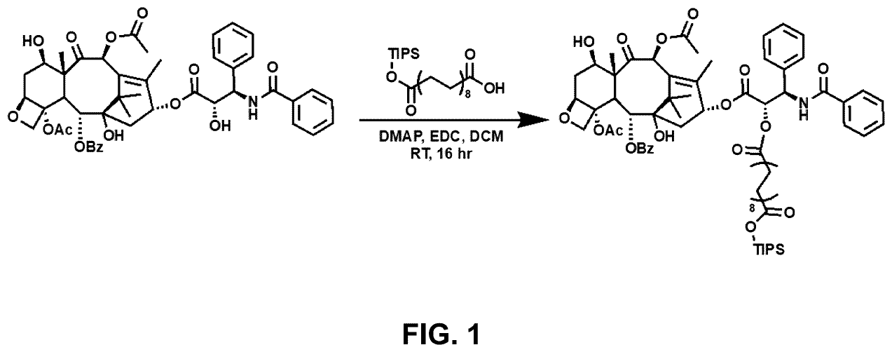

Synthesis of ODDA-TIPS-PTX: To a cold solution of ODDA-TIPS, (841 mg, 1.78 mmol) in 50 mL dichloromethane (DCM) was added 4-(dimethylamino)pyridine (DMAP, 614 mg, 2.97 mmol) and 5 minutes later, EDC (363 mg, 2.97 mmol). The resulting solution was stirred for 5 minutes, at which point PTX (1296 mg, 1.49 mmol) was added, and the reaction stirred overnight under N₂ and allowed to warm to room temperature. The exemplary schematic of FIG. 1 depicts this reaction. Reaction progress was monitored by thin layer chromatography (TLC) in 2:1 hexane:ethyl acetate. Upon complete consumption of PTX, the reaction was stopped and evaporated to dryness under vacuum by rotary evaporation. Product formation was confirmed by ¹H-NMR and ESI-MS (negative ion mode): MS-Theoretical=1147.59 [M-H]⁻; Observed=1148.38 [M-H]⁻.

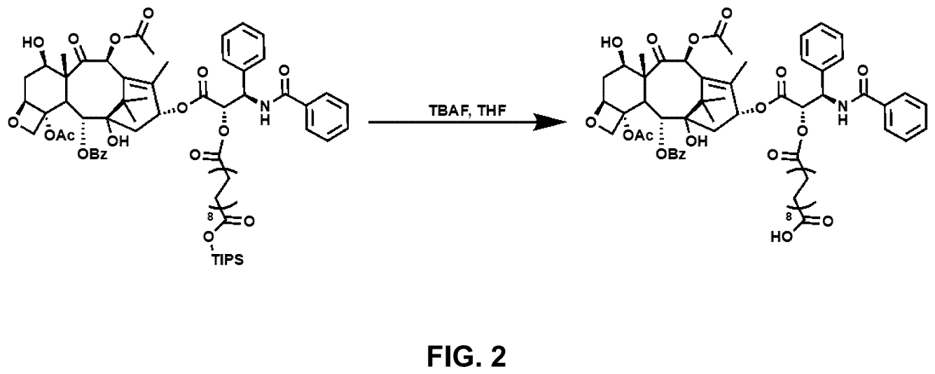

Deprotection of TIPS: As shown in the exemplary schematic of FIG. 2, tetrabutylammonium fluoride (TBAF) solution (1M in THF, 4.17 mmol) was added to a stirring solution of PTX-ODDA-TIPS (731 mg, 5.21 mmol) in THF (5 mL). The solution was stirred under nitrogen atmosphere for 4 hours, then concentrated to dryness. The crude was taken up into 10 mL of methanol, then 5 mL of MilliQ water was added and placed in the fridge overnight to precipitate the ODDA-PTX. The solution was filtered and dried after the 3 washes.

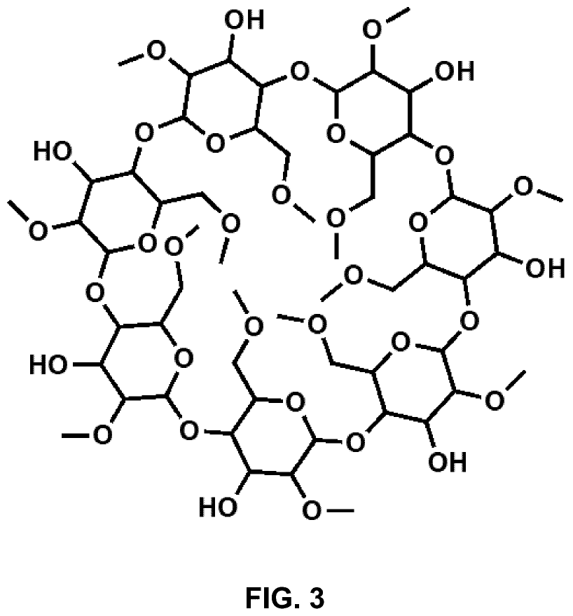

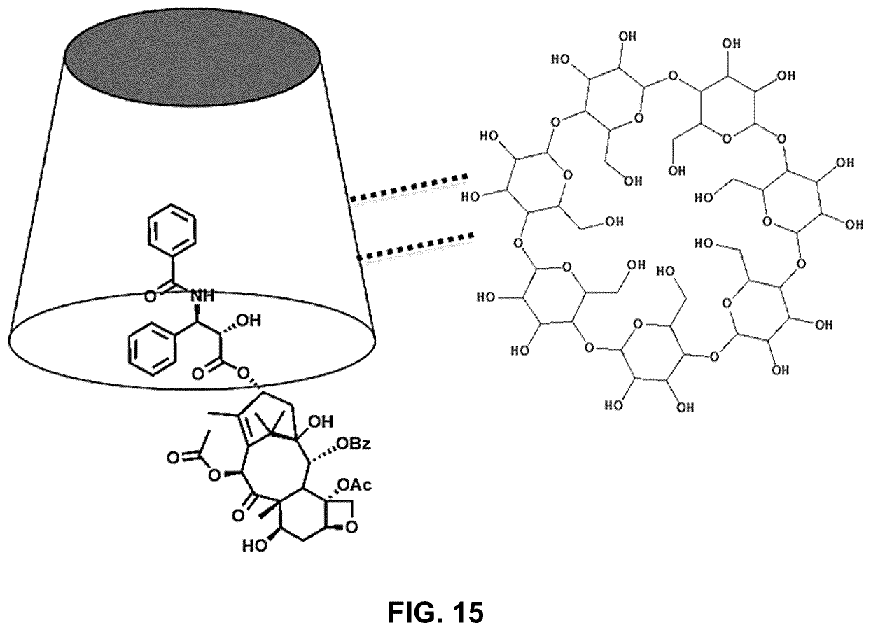

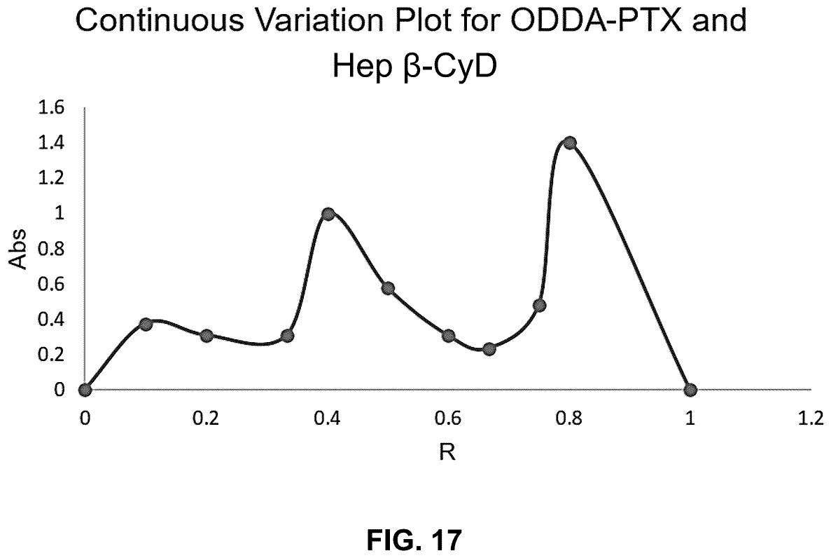

Formation of Inclusion Complex: ODDA-PTX (70 mg, 0.0609 mmol) was dissolved in 100 μL Acetonitrile and 400 μL tert-butanol and sonicated for 30 sec. Heptakis β-cyclodextrin (Hep β-CyD) (81.18 mg, 0.0609 mmol), the structure of which is portrayed in FIG. 3, was dissolved in 5 mL of MilliQ water and sonicated for 30 sec. The ODDA-PTX solution was added to the Hep β-CyD solution to create a milky, cloudy solution. The ODDA-PTX+Hep β-CyD solution was stirred overnight for 16 hr and filtered through a 0.45 μm filter and lyophilized to yield a fluffy, white powder. The same process was used to generate a mixture of ODDA-PTX and β-CyD. The materials were then characterized via ¹H-NMR in deuterium oxide. The ODDA-PX and β-CyD solution produced resonance peaks between 1 and 5.5 ppm corresponding to β-CyD's glucose hydrogen peaks. The ¹H-NMR spectra of ODDA-PTX+Hep β-CyD inclusion complex depicted the glucose hydrogen peaks of beta-cyclodextrin as well as the indicative peaks of paclitaxel phenyl groups (between 7.5 and 8.5). It is to be noted that due to the insolubility of paclitaxel in water, it is believed that the phenyl groups of paclitaxel appearing between 7.5-8.5 ppm are due to the complexation of the paclitaxel with Hep β-CyD, as uncomplexed material (i.e., a physical mixture) does not produce the same spectrum. These results suggest that the ODDA-PTX+Hep β-CyD inclusion complex maintains the structure of ODDA-PTX1n water. It is believed the methyl groups of Hep β-CyD (as shown in FIG. 3) attributed to this successful complexation, as similar experiments using underivatized β-CyD complexes (as shown in FIG. 15) did not produce peaks between 7.5-8.5 ppm. In view of these results, the ODDA-PTX+Hep β-CyD material was selected for additional experimentation. The material was further characterized via continuous variation to evaluate the complex stoichiometry for ODDA-PTX:Hep β-CyD, which was determined to be 3:7 (FIG. 17).

Solubility Tests: To verify ODDA-PTX aqueous solubility, characterization methods in milliQ water or dPBS were performed. The aqueous solubility of ODDA-PTX:Hep β-CyD was about 69.6 mM as determined by dissolving 10 mg of material in 1 mL and increasing by 10 mg intervals and sonicating for 30 seconds until reaching 80 mg of material. At 81 mg/mL, a suspension began to form. The ODDA-PTX:Hep β-CyD complex increased aqueous solubility of ODDA-PTX from 0.021 mg/ml to 10.91 mg/ml with respect to ODDA-PTX (a 500-fold increase). Considering paclitaxel has an aqueous solubility of less than 0.01 μM, the inclusion complex provides a 1000-fold improvement.

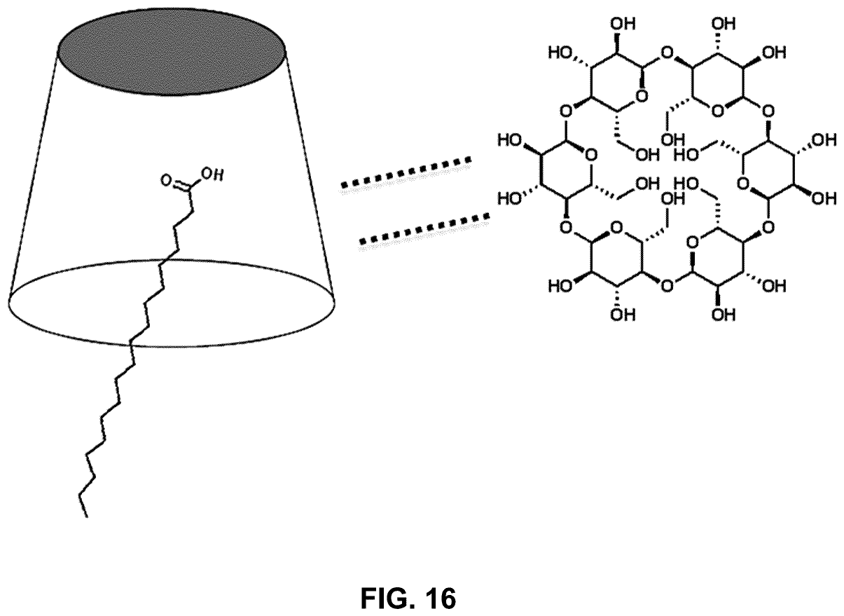

In a separate experiment using the same protocol as described in this Example 1, the formation of a ODDA-PTX:α-CyD inclusion complex (see e.g., FIG. 16) was attempted. However, when the ODDA-PTX solution was combined with aqueous α-cyclodextrin, the resulting solution could not be filtered through a 0.23 or a 0.45 μm sterile filter due to insolubility. Since sterile filtration is important for therapeutic injection purposes, β-CyD was selected for further experimentation.

### Example 2

An in vitro tubulin polymerization assay was conducted to confirm that the prodrug status of ODDA-PTX is maintained in the β-cyclodextrin inclusion formulation described in Example 1.

Tubulin protein (>99% pure, extracted from porcine brain) was reconstituted in buffer solution (80 mM PIPES pH 6.9, 2 mM MgCl₂, 0.5 mM EGTA, and 10% (v/v) glycerol) to a concentration of 1 mg/mL. 100 μL of this tubulin solution was added per well of a black, 96-well plate, which had been warmed to 37° C. prior to the experiment. This was followed by the addition of either guanosine triphosphate (GTP), Paclitaxel (PTX), or ODDA-PTX-CyD, where the final concentration in each respective well was 1 mM GTP, 10 μM PTX, or 10 μM ODDA-PTX-CyD. Changes in turbidity of the samples were monitored via 96-well plate reader at 350 nm, where a recorded increase in optical density supports the formation of microtubules in solution. Each set of conditions were measured in triplicate and monitored over 20 minutes at 37° C.

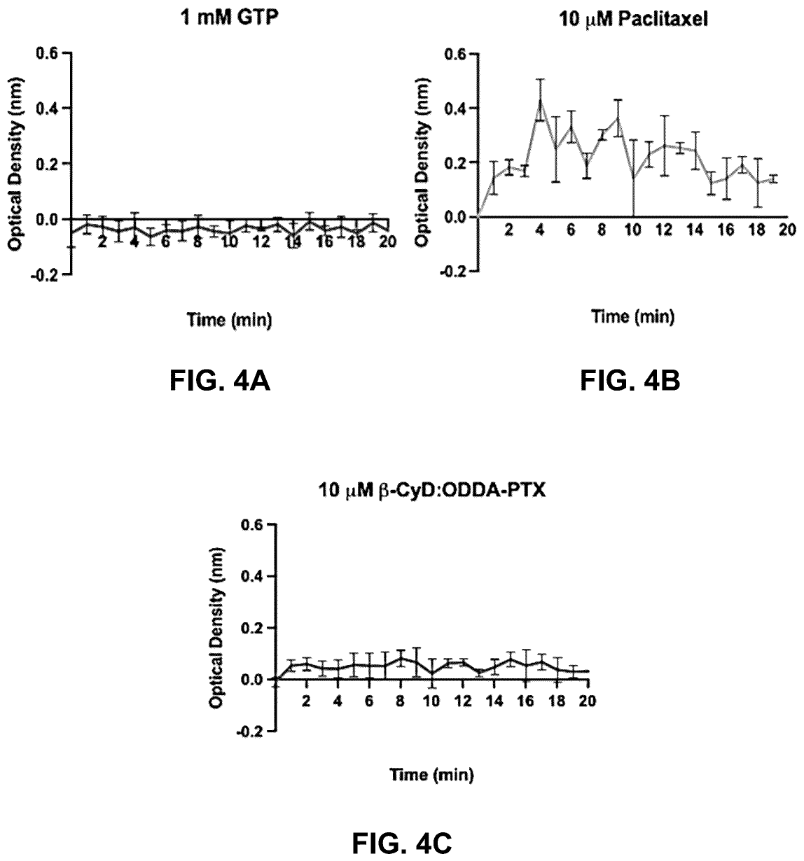

As shown in FIGS. 4A and 4C, the tubulin solution did not portray significant increases in optical density when combined with GTP (FIG. 4A) or ODDA-PTX-CyD (FIG. 4C). Unlike the tubulin solution treated with PTX alone, which resulted in the formation of microtubules (as shown in FIG. 4B), the tubulin did not react with PTX within the ODDA-PTX-CyD inclusion complex. These results confirmed that the ODDA-PTX:Hep β-CyD maintained prodrug status by not creating the tubulin dimers necessary for tubulin polymerization.

### Example 3

HPLC Analysis was run to evaluate the relative retention times of ODDA-PTX:Hep β-CyD, ODDA-PTX, and Hep β-CyD.

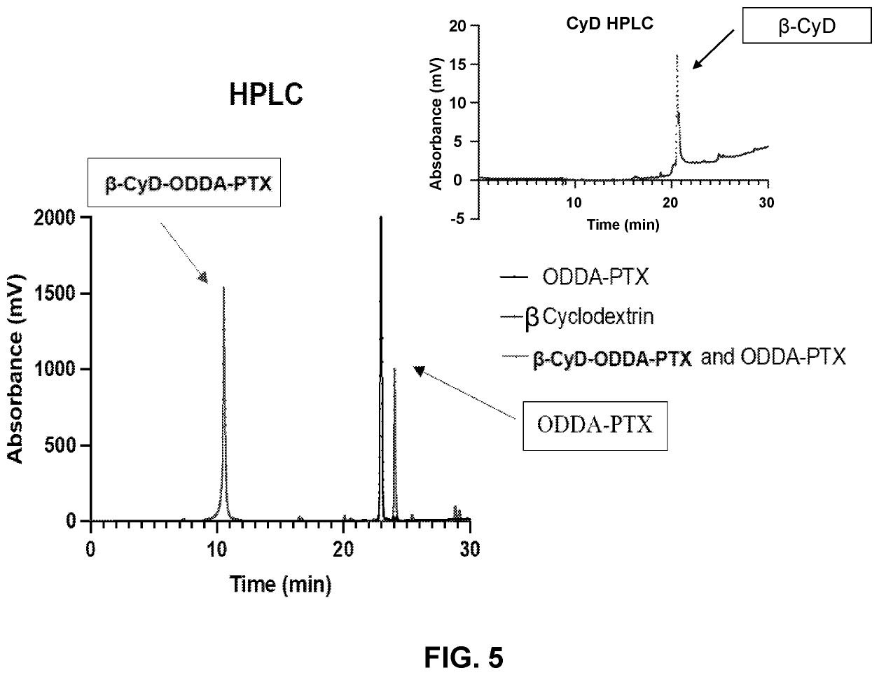

ODDA-PTX:Hep β-Cyclodextrin and ODDA-PTX at a 1:1 ratio were co-dissolved in 1 mL of 50% Buffer A (water+0.1% TFA) and 50% Buffer B (Acetonitrile+0.1% TFA). The sample was ran on a 0% to 100% Buffer B gradient to determine the change in elution time. β-Cyclodextrin was ran separately at the same concentration in 50% Buffer A and 50% Buffer B. The β-cyclodextrin formulation of ODDA-PTX had a shorter retention time, as shown in FIG. 5. The shorter retention time is a result of the increased solubility provided by the inclusion complex.

### Example 4

Preparation of ODDA-PTX:Hep β-CyD with HSA.

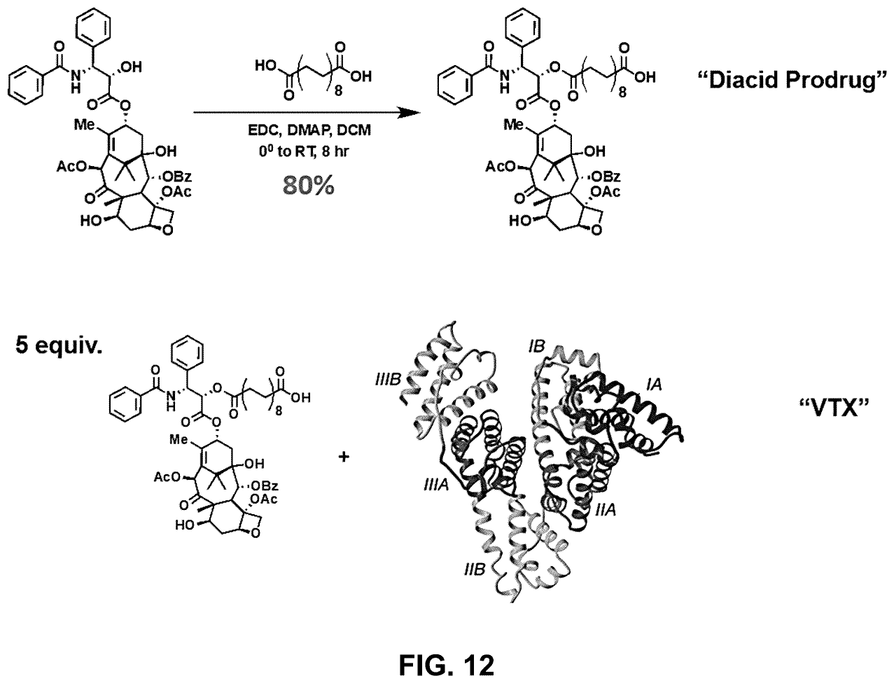

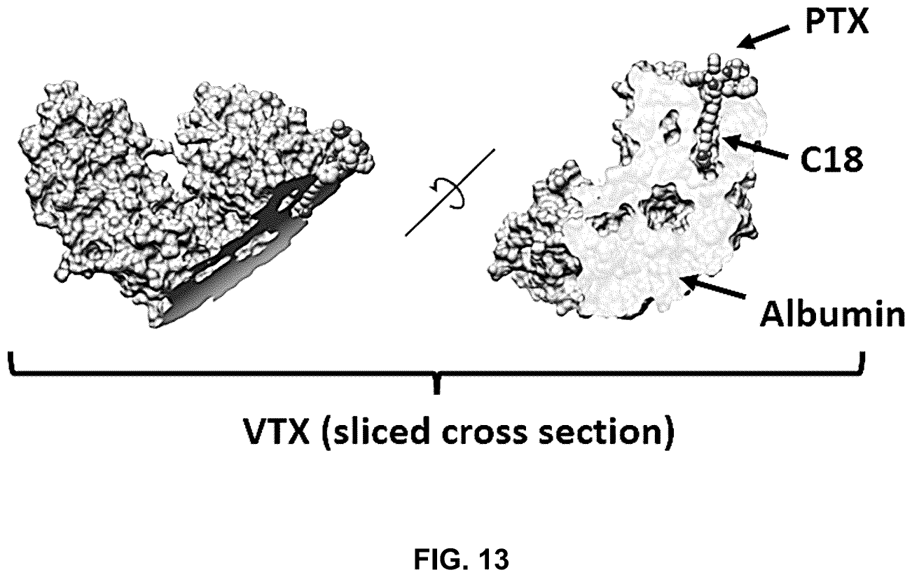

To prepare stock solutions, ODDA-PTX:Hep β-CyD was dissolved in MilliQ water at a concentration of 0.030075 M, and HSA (Sigma A1887, lyophilized powder, essentially fatty acid free) was dissolved in MilliQ water at a concentration of 0.0006015 M (40.0 mg/mL). To a 1 mL centrifuge tube, the clear colorless ODDA-PTX-Hep β-CyD stock solution was added, followed by rapid addition of 37.40 mL of the clear yellow HSA stock to generate a 5:1 mol ratio of ODDA-PTX:HSA solution. Solution was then vortexed for 10 seconds, snap frozen under liquid nitrogen, and lyophilized to afford a white powder (VTX). FIG. 12 provides an exemplary schematic of the preparation of ODDA-PTX (top, “Diacid Prodrug”) and the resulting mixture of ODDA-PTX with HSA (bottom, “VTX”). VTX was reconstituted as a clear and colorless solution in buffered water (DPBS, Dulbecco's Phosphate Buffered Saline, no Ca and no Mg) to a concentration of 25.0 mg/mL with respect to ODDA-PTX content. It is believed that this mixture of ODDA-PTX-Hep β-CyD results in ODDA binding to a fatty acid binding site of HSA as depicted in the exemplary schematic in FIG. 13.

### Example 5

This Example investigated whether the ODDA-PTX:β-CyD formulation had any effect on HSA protein folding.

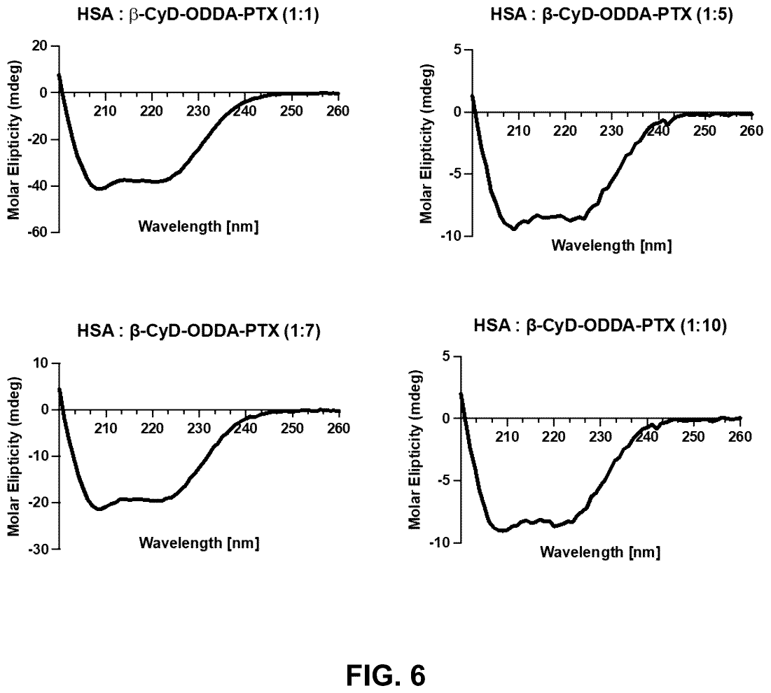

ODDA-PTX-Hep β-CyD was formulated with HSA at mol ratios of 1:1, 5:1, 7:1, and 10:1 ODDA-PTX:HSA in the same general manner as described in Example 4. Solutions were lyophilized overnight, and then resuspended in 1.0 mL DPBS and analyzed by CD to probe the structure of HSA as a function of bound ODDA-PTX-Hep CyD. No changes in molar ellipticity were observed for any mol ratio tested in the CD results, as shown in FIG. 6, indicating no change in HSA folding.

### Example 6

Once it was verified that the ODDA-PTX-Hep β-CyD did not impact HSA folding, the inclusion complex was investigated for its binding capabilities with HSA. The binding of ODDA-PTX-βCyD to HSA was investigated through isothermal titration calorimetry (ITC) and ¹³C-palmitic acid displacement via NMR analysis.

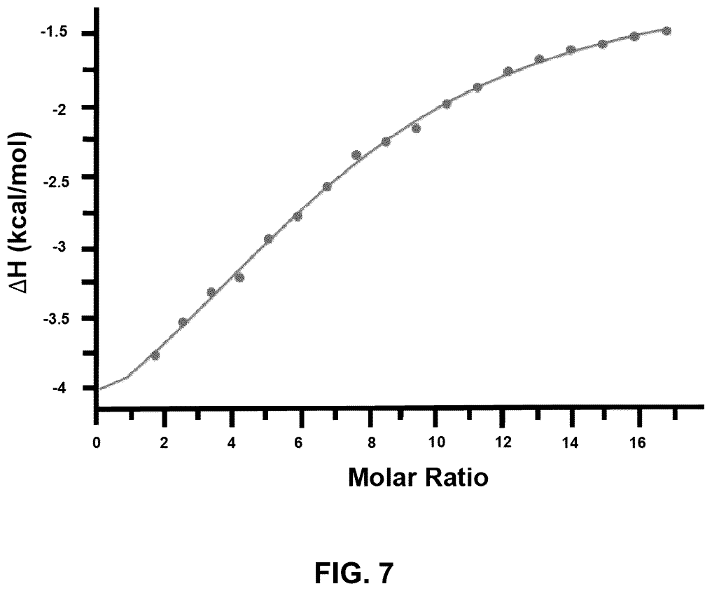

ITC: To probe protein binding using ITC, ODDA-PTX-Hep β-CyD was dissolved at 47.9 mM in DPBS buffer. HSA was dissolved in DPBS buffer at a concentration of 4.57 mM, followed by degassing under vacuum. ITC was performed using an Affinity ITC from TA instruments. The ODDA-PTX-Hep β-CyD solution was loaded into the syringe and HSA in the sample cell. The temperature was set to 25° C., with 2 μL of the ODDA-PTX-Hep β-CyD solution injected every 180 sec. The reference power was set to 10 μCal/sec, with an initial delay of 60 seconds and a stirring speed of 120 rpm. The ODDA-PTX-Hep β-CyD was found to have a dissociation constant of 62 μM and is capable of binding to all 7 fatty acid binding sites on HSA (FIG. 7).

¹³C-palmitic acid displacement: First, a stock solution of ¹³C-palmitic acid mixed with HSA at a 5:1 mol ratio (palmitic acid:HSA) was prepared in 1:1 D₂O:H₂O at 30.0 mg/mL with respect to HSA. This solution was prepared by transferring a known amount of palmitic acid to a vial, then adding the appropriate volume of HSA in solution and stirring overnight at room temperature to allow the palmitic acid to associate with HSA and enter solution. After overnight incubation, a clear off-yellow solution was observed. Next, 1.0 mL of the HSA/palmitic acid conjugate was removed to a vial containing 6.662 mg ODDA-PTX-Hep β-CyD (to produce a 30:1 mol ratio of ODDA-PTX-Hep β-CyD:HSA). This new solution was equilibrated overnight. Both samples (palmitic acid/HSA and ODDA-PTX-CyD/HSA) were analyzed by ¹³C-NMR to look for the disappearance of peaks characteristic of palmitic acid binding to HSA. The palmitic acid/HSA sample showed sustained rapid displacement, and ODDA-PTX-Hep β-CyD/HSA showed similar displacement at varying concentrations.

### Example 7

In this Example, cell viability was assessed after treatment with ODDA-PTX:Hep β-CyD/HSA (VTX).

Cytotoxicity of ODDA-PTX-Hep β-CyD/HSA (VTX) was evaluated using the CellTiter Blue (CTB) assay (Promega, cat G8081). The ODDA-PTX-Hep β-CyD was prepared as a concentrated solution in DPBS, then diluted in media for treatment solutions. Fibrosarcoma cells (HT-1080) and adenocarcenoma cells (MCF-7) were plated in 96-well plates, 1 day prior to treatment, at the following densities: HT-1080 at 4,500 cells/well and MCF-7 at 10,000 cells/well. After 24 hours, plating media was removed, then treatments of 100 μL were added to the wells, at concentrations ranging from 0.1 nM to 10 μM with respect to ODDA-PTX, three technical repeats per treatment concentration. After 3 days, the media was removed and replaced with complete DMEM without phenol red. At this point, 20 μL of CTB reagent was added, and the cells incubated for 4 hours at 37° C. Fluorescence was measured at 590 nm with excitation at 560 nm using a Perkin Elmer EnSpire plate reader. The average background fluorescence of CTB in media without cells was subtracted from the average fluorescence readings of the experimental wells (three wells per treatment concentration). Viability was calculated as the average background-subtracted signal in a well compared to that of a negative control well (treatment with vehicle, either 0.1% DMSO/media or media). Viabilities were fit in GraphPad Prism using a non-linear, dose-dependent inhibition curve. Values for IC₅₀ reflect the concentration at which cell death is 50% of the maximum response. The log (IC₅₀) and error in logic are reported and reflect the standard error in the fit.

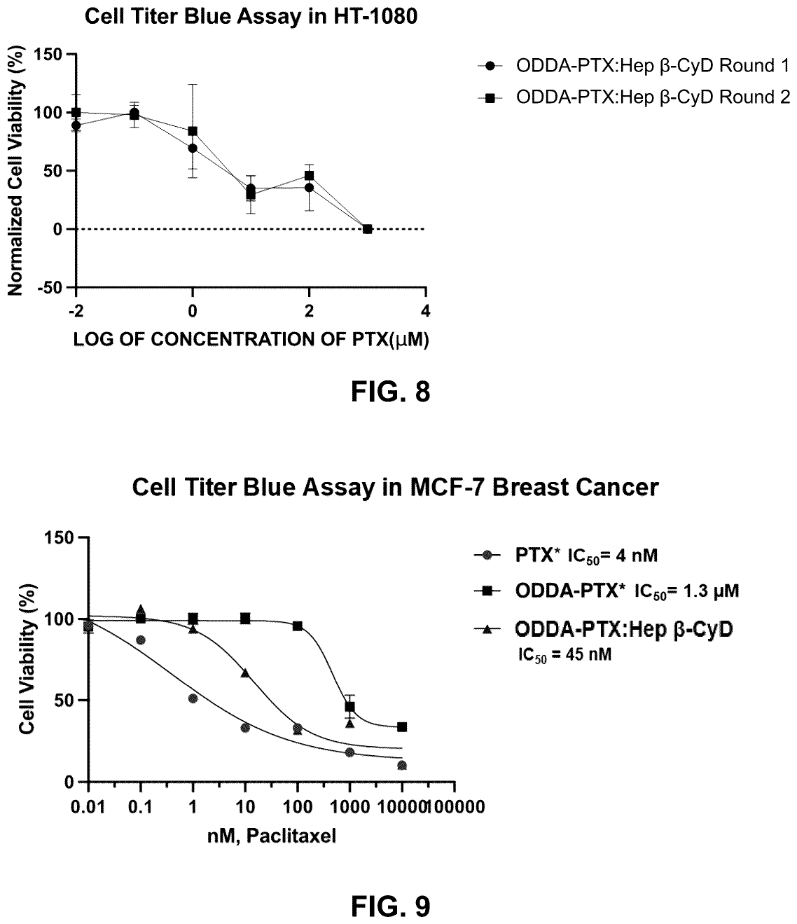

As shown in FIG. 9, the cyclodextrin complex of ODDA-PTX had an IC₅₀ value of 0.869 μM in the MCF-7 model. In the HT-1080 model, the inclusion complex reflected an IC₅₀ value of 9.997 μM (FIG. 8).

### Example 8

To further characterize the ODDA-PTX-Hep β-CyD three-dimensional images of the complex in solution were taken and observe.

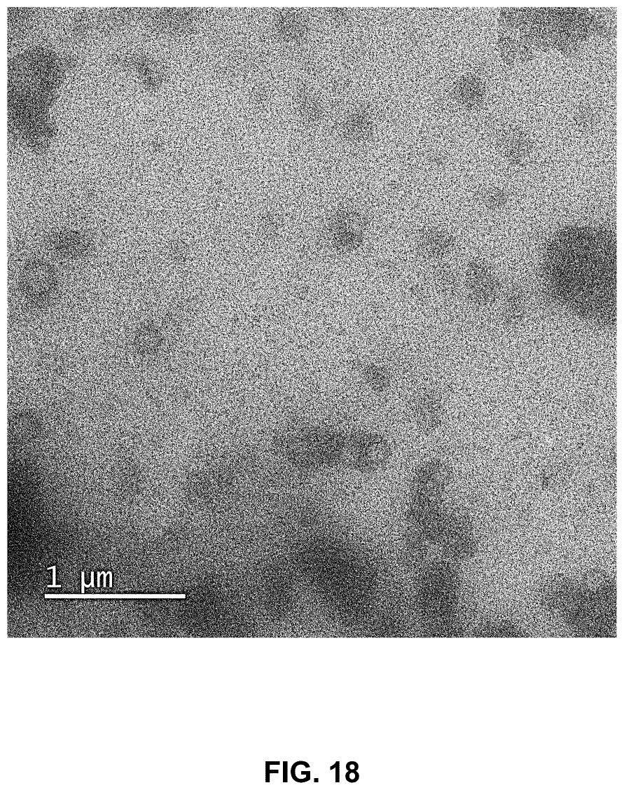

20 μL of samples were applied onto a 400 mesh carbon grids (Ted Pella, INC.). The grids were observed on a Hitachi HT 7700 microscope operating at 12 kV. The images were recorded with a slow-scan charge coupled device (CCD) camera (Veleta 2k×2k). As shown in FIG. 18, the ODDA-PTX-Hep β-CyD, when in a solvated state, results in micelle formation assembling into small aggregates. It is believed that the ratio of cyclodextrin to ODDA-PTX contributes to these results. Specifically, it is believed that cyclodextrin forms electrostatic interactions with the PTX warhead. Due to the lipophilicity of the fatty acid (i.e., ODDA), the lipids create the hydrophobic core while the hydrophilic PTX is exposed to the exterior.

### Example 9

Based on the results of Examples 1-8, efficacy of the inclusion complex in treating fibrosarcoma was evaluated in vivo.

Nu/nu mice were inoculated with ˜10⁶ HT-1080 cells on the right flank, and treatments were initiated when the average animal tumor burden was between 50 and 150 mm³. Dosing was performed via tail vein (IV) administration of VTX, saline, Abraxane, or crPTX, once a week for four weeks. Body weights were measured daily as well as tumor size via caliper examination. Animals were analyzed for survival by monitoring for tumor burden for the entire cohort. Dosing was at both 20 and 250 mg/kg for VTX and was 15 mg/kg for crPTX and Abraxane (known MTD). crPTX refers to the Cremaphor EL pharmaceutical formulation of Paclitaxel.

The therapeutic index (TI) was defined as the ratio of maximum tolerated dose (MTD) to minimum effective dose (MED) where MTD was the highest nonlethal deliverable dose and less than 20% weight loss, and MED was the lowest dose showing a delay in tumor growth relative to nontreated controls. Efficacy of VTX was investigated in the HT-1080 model following intravenous (IV) injection at 5, 60, 120, and 250 mg/kg with respect to PTX concentration every 7 days for 4 weeks (q7dx4). Animals responded to VTX1n a dose dependent manner. Complete tumor regression (5/8) or dramatically suppressed progression was observed in animals administered VTX at 250 mg/kg, with no evidence of drug-associated toxicity observed. Conversely, animals administered Abraxane at 20 mg/kg with respect to PTX concentration experienced significant treatment-associated lethality. Therefore, the highest dose of Abraxane administered in this model was limited to 15 mg/kg. We note that these MTDs are lower than what is reported in several published studies. Similar antitumor activity of crPTX at 15 mg/kg was observed to that of Abraxane at 15 mg/kg, the reported MTD of crPTX.

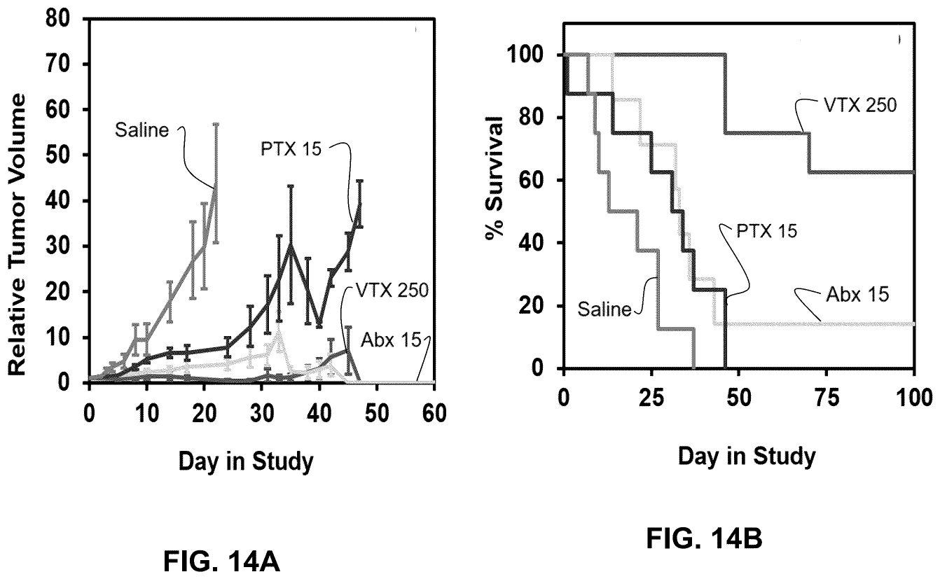

Elevated doses of VTX significantly extended survival time and inhibited tumor growth over nontreated controls, whereas the improvement of survival time for Abraxane and crPTX was modest (FIG. 14A depicting relative tumor growth; FIG. 14B depicting survival time). The lowest administered dose (5 mg/kg) of VTX and Abraxane had similar effects on median survival time and both treatments sufficiently suppressed tumor growth relative to nontreated controls over the course of the study (p-value=0.041 and 0.045, respectively, based on an unpaired t test where p<0.05 was considered statistically significant). This was defined as the MED for both VTX and Abraxane in this model and with this dosing regimen. In evaluating the efficacy in tumor growth suppression and survival of animals treated with VTX and Abraxane, we conclude a distinct advantage of VTX over the FDA-approved drug. In this model, VTX had a MED of 5 mg/kg and a MTD of>250 mg/kg with respect to PTX concentration. Higher doses could not be explored due to solubility constraints of HSA used in the formulation of VTX. Thus, the TI of VTX is>50 in the HT-1080 model at a q7dx4 dosing regimen. Conversely, Abraxane-treated animals experienced significant toxicity at all doses exceeding 15 mg/kg, revealing a TI of 3 for Abraxane.

From the information gathered from Examples 1-9, the Hep β-CyD-ODDA-PTX complex maintains bioactivity in saline and improves aqueous solubility of paclitaxel, specifically from 0.021 mg/ml to 10.91 mg/ml with respect to ODDA-PTX. The β-CyD-ODDA-PTX complex demonstrated the ability to bind with HSA without impacting HSA folding. Moreover, the water-soluble, albumin-free formulation allows for the exploration of the MTD for ODDA-PTX without the limitations of albumin solubility. Similarly, the lack of protein in the formulation allows this inclusion complex to be amended to combination therapies, which is the typical mode of treatment for most cancers.

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An inclusion complex characterized by formula (FX1):
(A¹—X¹—X²—T²)m:(β-CyD)n (FX1)
or a salt thereof,
wherein:
A¹ is a carboxylic acid group, a carboxylate anion, or a carboxylate ester;
T² is a cytotoxic taxane drug moiety, which has a molecular weight of no more than 1600 Da;
X¹ is an optionally substituted alkylene group having 8-22 carbon atoms;
X² is a direct bond, an organic group moiety, —O—C(═O), —O—C(═O)—O—, —C(═O)—, —O—, —S—, —S(═O)—, —S(═O)₂—, —S—S—, —N═, ═N—, —N(H)—, —N═N—N(H)—, —N(H)—N═N—, —N(OH)—, or —N(═O)—;
β-CyD is a water-soluble beta-cyclodextrin;
m is the number of derivatized cytotoxic taxanes associated with the beta-cyclodextrin in the inclusion complex; and
n is the number of beta-cyclodextrin associated with the derivatized cytotoxic taxane drug moiety in the inclusion complex;
wherein the ratio of m:n ranges from 1:2 to 3:10.

2. The inclusion complex of claim 1, wherein T² is a paclitaxel moiety, a docetaxel moiety, a cabazitaxel moiety, a larotaxel moiety, an ortataxel moiety, a tesetaxel moiety, or a milataxel moiety.

3. The inclusion complex of claim 1, wherein X¹ is an unsubstituted alkylene group having 8-22 carbon atoms.

4-6. (canceled)

7. The inclusion complex of claim 1, wherein β-CyD is a derivatized β-CyD.

8-9. (canceled)

10. The inclusion complex of claim 1, wherein β-CyD is a heptakis beta-cyclodextrin, a hetakis beta-cyclodextrin, a randomly methylated beta-cyclodextrin, or a 2-hydroxypropyl-beta cyclodextrin.

11. (canceled)

12. The inclusion complex of claim 1, wherein the molar ratio of T² to β-CyD is between 1:1 and 1:10.

13-16. (canceled)

17. The inclusion complex of claim 1, wherein T² is a paclitaxel moiety, wherein the paclitaxel moiety is characterized by formula (FX2):

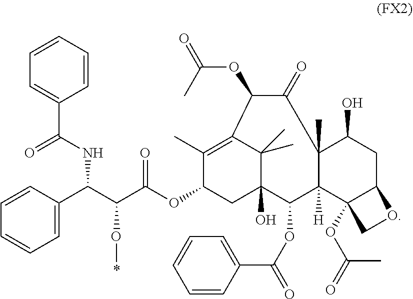 [SMILES: *O[C@@H](C(=O)O[C@H]1C[C@@]2(O)[C@@H](OC(=O)c3ccccc3)[C@H]3[C@](C)(CC(=O)[C@H](OC(C)=O)C(=C1C)C2(C)C)[C@@H](O)C[C@H]1OC[C@]13OC(C)=O)[C@@H](NC(=O)c1ccccc1)c1ccccc1]

18. An aqueous pharmaceutically acceptable solution comprising:
an aqueous carrier; and
one or more than one inclusion complex or salt thereof of claim 1, wherein the total concentration of the cytotoxic taxane drug moiety in the aqueous solution is equal to 0.5 mg/mL or higher.

19-21. (canceled)

22. The aqueous pharmaceutically acceptable solution of claim 18, wherein the aqueous pharmaceutically acceptable solution does not include albumin.

23. The aqueous pharmaceutically acceptable solution of claim 18, wherein the aqueous pharmaceutically acceptable solution does not include proteins.

24. The aqueous pharmaceutically acceptable solution of claim 18, wherein the aqueous pharmaceutically acceptable solution does not include one or more of separately added liposomes, polymers, surfactants, dimethyl sulfoxide, ethanol, or ethylene glycol.

25-30. (canceled)

31. The aqueous pharmaceutically acceptable solution of claim 18, wherein the aqueous carrier is sterile water for injection or pharmaceutically acceptable saline, wherein the aqueous carrier does not comprise an organic solvent.

32. (canceled)

33. The aqueous pharmaceutically acceptable solution of claim 18, further comprising one or more of an immunomodulating agent.

34-36. (canceled)

37. An aqueous pharmaceutically acceptable solution comprising:
a water soluble beta-cyclodextrin; and
one or more derivatized cytotoxic taxane drug moiety of formula (FX1a):
A¹—X¹—X²—T² (FX1a)
or a salt thereof,
wherein:
A¹ is a carboxylic acid group, a carboxylate anion, or a carboxylate ester;
T² is a cytotoxic taxane drug moiety, which has a molecular weight of no more than 1600 Da;
X¹ is an optionally substituted alkylene group having 8-22 carbon atoms; and
X² is a direct bond, an organic group moiety, —O—C(═O), —O—C(═O)—O—, —C(═O)—, —O—, —S—, —S(═O)—, —S(═O)₂—, —S—S—, —N═, ═N—, —N(H)—, —N═N—N(H)—, —N(H)—N═N—, —N(OH)—, or —N(═O)—;
wherein the total concentration of the one or more derivatized cytotoxic taxane drug moiety in the aqueous pharmaceutically acceptable solution is equal to 0.5 mg/mL or higher.

38. The aqueous pharmaceutically acceptable solution of claim 37, wherein the molar ratio of the derivatized cytotoxic taxane drug moiety to the water soluble beta-cyclodextrin ranges from 1:1 to 3:10.

39. The aqueous pharmaceutically acceptable solution of claim 37, wherein the derivatized cytotoxic taxane drug moiety and the water soluble beta-cyclodextrin are substantially in the form of an inclusion complex.

40. A method of treating cancer, the method comprising administering to a subject a therapeutically effective amount of the aqueous pharmaceutically acceptable solution of claim 18.

41. (canceled)

42. The method of claim 40, wherein the cancer comprises cells that overexpress one or more fatty acid uptake proteins.

43. The method of claim 40, wherein the cancer is breast cancer, ovarian cancer, lung cancer, Kaposi's sarcoma, fibrosarcoma, prostate cancer, or pancreatic cancer.

44. The method of claim 40, wherein the subject is a neonate, an infant, a child, or an adolescent.

45. The method of claim 40, wherein the aqueous pharmaceutically acceptable solution is administered to the subject in combination with one or more additional therapeutic agents.

46-50. (canceled)

51. The method of claim 40, further comprising the step of: pre-mixing the aqueous pharmaceutically acceptable solution with human serum albumin prior to the administering step.

52. The method of claim 51, wherein the molar ratio of cytotoxic taxane drug moiety to human serum albumin ranges from 1:1 to 10:1.

53. (canceled)

54. A method for preparation of an improved pharmaceutically acceptable aqueous solution containing 9.5 mM or greater of a cytotoxic taxane moiety, which comprises the steps of:
a. preparing the inclusion complex of claim 1 by mixing A¹—X¹—X²—T² with β-CyD wherein the molar ratio of β-CyD to A¹—X¹—X²—T² is greater than 1 to form one or more inclusion complexes; and
b. dissolving the one or more inclusion complexes in a selected pharmaceutically acceptable aqueous solvent at a concentration equal to or greater than 9.5 mM with respect to the cytotoxic taxane drug moiety.

55. A method of generating the inclusion complex of claim 1, the method comprising the steps of:
a. providing a solvent;
b. dissolving A¹—X¹—X²—T² in the solvent to form a solution;
c. preparing an aqueous β-CyD by dissolving β-CyD in water;
d. combing the aqueous β-CyD and the solution wherein the molar ratio of β-CyD to A¹—X¹—X²—T² is greater than 1 to form a mixture; and
e. removing the solvent from the mixture;
wherein the removing step results in the formation of a solid, thereby generating the inclusion complex.

56. (canceled)

57. The method of claim 55, further comprising the step of:
filtering the mixture prior to the step of removing the solvent from the mixture.

58-59. (canceled)

60. The method of claim 55, further comprising reconstituting the solid with a pharmaceutically acceptable aqueous carrier to form an aqueous solution at a concentration equal to or greater than 6.8 mM with respect to the cytotoxic taxane drug moiety.

61. The method of claim 60, wherein the pharmaceutically acceptable aqueous carrier is one or more of phosphate buffered saline, tris buffered saline, saline solution, sterile water for injection, or sterile saline for injection, wherein the pharmaceutically acceptable aqueous carrier does not include an organic solvent.

62. (canceled)
